(19) United States
(12) Patent Application Publication
(10) Pub. No.: US 2015/0250791 A1
(43) Pub. Date: Sep. 10, 2015

(54) COMBINING SILDENAFIL WITH CAFFEINE IN AN ORAL DISINTEGRATING DOSAGE FORM

(71) Applicant: BHASKARA RAO JASTI, STOCKTON, CA (US)

(72) Inventors: ABDEL HALEM SA, CAIRO (EG); EL-NABARAWI MOHAMMED, CAIRO (EG); EL-RAFAAI KI, CAIRO (EG); BHASKARA RAO JASTI, STOCKTON, CA (US)

(21) Appl. No.: 14/462,518

(22) Filed: Aug. 18, 2014

Related U.S. Application Data
(60) Provisional application No. 61/966,899, filed on Mar. 6, 2014.

(51) Int. Cl. A61K 31/522; A61K 31/519

(52) U.S. Cl. CPC A61K 31/522; A61K 31/519

(57) ABSTRACT

Pharmaceutical compositions and formulations are provided herein comprising a phosphodiesterase type 5 inhibitor, such as sildenafil citrate; and, an adenosine receptor (A1, A2A, A2B, and A3 receptors) antagonist, such as caffeine, for (i) treating erectile dysfunction while (ii) inhibiting the lower of the blood pressure, (iii) increasing the bioavailability of the compositions, and (iv) reducing the Tmax in the subject. Methods of making and administering oral disintegrating tablets are also provided.

### CROSS-REFERENCE TO RELATED APPLICATIONS

This application claims the benefit of U.S. Provisional Application No. 61/966,899, filed Mar. 6, 2014, which is hereby incorporated herein in its entirety by reference.

### BACKGROUND

1. Field of the Invention

The teachings provided herein relate to pharmaceutical compositions comprising a phosphodiesterase type 5 inhibitor and caffeine for treating erectile dysfunction rapidly in a subject while inhibiting the lower of the blood pressure of the subject

2. Description of Related Art

Erectile dysfunction, otherwise known as “ED”, is a widespread condition with a negative impact on the quality of life. It has been estimated that at least 20 million American men suffer erectile dysfunction, and this can be a total inability to achieve erection, an inconsistent ability to achieve an erection, or a tendency to sustain only a brief erection. The disorder increases with age, affecting about 5% of men at around the age of 40, and between 15-25% of men at around the age of 65.

Sildenafil citrate (sildenafil) was discovered by Pfizer and filed as a patent application on Jun. 20, 1990, as a treatment of various cardiovascular disorders such as angina, hypertension, heart failure and atherosclerosis. It was approved for use in erectile dysfunction by the FDA on Mar. 27, 1998, becoming the first oral treatment approved to treat erectile dysfunction in the United States, treating erectile dysfunction by inhibiting the enzyme PDE5. Inhibiting this enzyme allows the cyclic GMP (cGMP) to stay-around longer, thus maintaining an erection. Many physiological disorders can affect the ability to achieve an erection, and sildenafil has proven to be an effective therapy. Clinical trials in patients with vascular diseases, diabetes mellitus, spinal cord injuries, psychogenic causes, and after radical prostrate surgery have shown that 70%-80% of men reported improved quality of their erections, and over 50% of men were able to have successful vaginal penetration.

Unfortunately, sildenafil is not without problems, one of which is the onset of hypotension. For example, some men are unable to take sildenafil without substantial risk, and some are unable to take it at all, as sildenafil is known to cause a drop in blood pressure. It is not recommended for use with nitrates that are used to wide arteries including, for example, nitroglycerin, isosorbide dinitrate, sorbitrate, isosorbide mononitrate, amyl nitrite or amyl nitrate poppers, and the like. As such, men with cardiovascular disease should take special precautions, and some cannot use it under any circumstances. It should be appreciated that, due to the very high prevalence of heart disease, this is a significant problem that prevents the use of sildenafil by those that could otherwise enjoy it's benefits. Even a control group of healthy volunteers given sildenafil followed an hour later by nitroglycerin have shown blood pressures drops of 25-51 mm Hg, which can be dangerous. A work-around has been to stop use of nitrates before taking sildenafil, but this sets-forth a serious risk for men suffering heart disease, as this practice prevents the use of nitrates for at least 24 hours and, in some cases, 48 hours. The FDA, for example, has urged caution in patients who have suffered heart attacks, strokes, or serious disturbances of the heart's pumping rhythm in the previous six months, in men with a history of congestive heart failure or unstable angina, and in men with low blood pressure or uncontrolled high blood pressure (above 170/110 mm Hg).

A second problem is the low bioavailability of sildenafil, about 40%, as it is currently administered orally for absorption through the digestive tract. Sildenafil is typically administered orally one-hour before sexual activity is expected, and preferably on an empty stomach, as it is currently absorbed through the digestive tract. As such, the effectiveness of the drug can vary significantly due to stomach contents, for example, the presence of a fatty meal. Oral bioavailability of sildenafil is low when compared to an intravenous administration, for example, as about 80% of the sildenafil absorbed through the digestive tract is metabolized by CYP3A4 in the liver to a less active compound, N-desmethyl sildenafil. The art has still not provided an acceptable dosage form that bridges-the-gap between the low bioavailability achieved through digestive administration and the bioavailability obtained through the rapid, systemic intravenous administration. A desirable dosage form would provide ease of administration for the user while bypassing the deleterious effects of the digestive tract on the pharmacokinetics of the dosage form.

A third problem is the increase in time (Tmax) to maximum concentration (Cmax) of sildenafil in a subject due to the limitations and variations present due to reliance on gastrointestinal absorption. In addition to the bioavailability issues discussed above, the presence of food in the stomach can have a dramatic effect on Tmax. The art has observed statistically significant differences have been seen between fasted and fed states for the Cmax and Tmax with sildenafil, in that the Cmax of sildenafil in fed subjects has been shown to be about 70% of that in fasting subjects. Fasting subjects have a Tmzx average of about 1 hr, and the presence of food in the stomach has been shown to delay the mean Tmax by about 1.1 hr.

Accordingly, and for at least the above reasons, one of skill in the art will appreciate having a new formulation for (i) treating erectile dysfunction in a subject while (ii) inhibiting a lowering of blood pressure, so that more people can benefit; and a dosage form of the new composition that bypasses absorption through the digestive tract to (lip increase the bioavailability of the new composition; and (iv) reduce the Tmax in the subject.

### SUMMARY

Pharmaceutical compositions and formulations are provided herein comprising a phosphodiesterase type 5 inhibitor, such as sildenafil citrate; and, an adenosine receptor (A1, A2A, A2B, and A3 receptors) antagonist, such as caffeine, for (i) treating erectile dysfunction while (ii) inhibiting the lower of the blood pressure, (iii) increasing the bioavailability of the compositions, and (iv) reducing the Tmax in the subject. Methods of making and administering oral disintegrating tablets are also provided.

As such, compositions are provided that comprise a phosphodiesterase type 5 inhibitor (PDE5 inhibitor), or a pharmaceutically acceptable salt thereof, and caffeine, wherein the composition treats erectile dysfunction in a subject while inhibiting a lowering of blood pressure in a subject. In some embodiments, the PDE5 inhibitor is sildenafil citrate.

Pharmaceutical formulations are provided comprising such a composition, wherein the PDE5 inhibitor is sildenafil citrate; and, the pharmaceutical formulation is in the form of an oral disintegrating tablet designed for a primary absorption through buccal or sublingual mucosa. The tablet can have a matrix former, a sugar alcohol, and a collapse protectant; and, the oral disintegrating tablet provides a relative bioavailability value for the sildenafil citrate that is substantially greater than a tablet designed for a primary absorption through gastrointestinal mucosa.

In some embodiments, the matrix former can be selected from the group consisting of gelatin, xanthan gum, Na-carboxymethyl cellulose, and AEROSIL200. In some embodiments, the sugar alcohol can be selected from the group consisting of mannitol, erythritol, sorbitol, trehalose, xylitol, glucose and sucrose. In some embodiments, the collapse protectant is selected from the group consisting of gelatin and glycine.

The pharmaceutical formulations can further comprise a solubilizer selected from the group consisting of polyethylene glycol, polyvinylpyrrolidone, and polysorbate; wherein, the dissolution rate of the sildenafil citrate in the subject being substantially higher than that of a control group receiving the sildenafil citrate without the solubilizer. In some embodiments, the solubilizer is polyethylene glycol 6000. In some embodiments, the solubilizer is polyvinylpyrrolidone K30. And, in some embodiments, the solubilizer is polysorbate 80.

The pharmaceutical formulations can further comprise a disintegrant selected from the group consisting of croscarmellose sodium, crospovidone, and sodium starch glycoate; wherein, the time to a maximum plasma concentration of the sildenafil citrate in the subject being substantially faster than that of a control group receiving the sildenafil citrate through a commercially available oral tablet configured for a primary absorption through gastrointestinal mucosa.

The pharmaceutical formulations can further comprise (i) a solubilizer selected from the group consisting of polyethylene glycol, polyvinylpyrrolidone, and polysorbate; and, (ii) a disintegrant selected from the group consisting of croscarmellose sodium, crospovidone, and sodium starch glycoate; wherein, the matrix former is selected from the group consisting of gelatin, xanthan gum, Na-carboxymethyl cellulose, and AEROSIL200; the sugar alcohol is selected from the group consisting of mannitol, erythritol, sorbitol, trehalose, xylitol, glucose and sucrose; and, the collapse protectant is selected from the group consisting of gelatin and glycine.

It should be appreciated that articles of manufacture is also provided, the article of manufacture comprising any composition or pharmaceutical formulation taught herein; and, instructions for administering an effective amount of the pharmaceutical formulation to a subject.

Methods of treating erectile dysfunction in a subject are also provided using any of the compositions, formulations, or articles of manufacture taught herein. The methods can inhibit a reduction in blood pressure of the subject while treating the erectile dysfunction. In some embodiments, the time to a maximum plasma concentration (Tmax) of an active agent is substantially faster than that of a control group receiving the active agent through a commercially available oral tablet configured for a primary absorption through gastrointestinal mucosa. And, in some embodiments, the bioavailability of an active agent in the subject is substantially higher than that of a control group receiving the active agent through a commercially available oral tablet configured for a primary absorption through gastrointestinal mucosa.

Methods of making an oral disintegrating tablet for treating erectile dysfunction through a buccal or sublingual absorption are also provided. The methods can include combining an effective amount of PDE5 inhibitor with an effective amount of caffeine to create an agent mixture; adding a matrix former, a sugar alcohol, and a collapse protectant to the sildenafil citrate and the caffeine; and, forming an oral disintegrating tablet that functions to deliver the agent mixture through a buccal or sublingual absorption. In some embodiments, the PDE5 inhibitor is sildenafil citrate. In some embodiments, the method further includes adding a disintegrant. In some embodiments, the method further includes adding a solubilizer. In some embodiments, the methods further comprise adding a solubilizer selected from the group consisting of polyethylene glycol, polyvinylpyrrolidone, and polysorbate; and, adding a disintegrant selected from the group consisting of croscarmellose sodium, crospovidone, and sodium starch glycoate; wherein, the matrix former is selected from the group consisting of gelatin, xanthan gum, Na-carboxymethyl cellulose, and AEROSIL200; the sugar alcohol is selected from the group consisting of mannitol, erythritol, sorbitol, trehalose, xylitol, glucose and sucrose; and, the collapse protectant is selected from the group consisting of gelatin and glycine.

One of skill reading the teachings that follow will appreciate that the concepts can extend into additional embodiments that go well-beyond a literal reading of the claims, the inventions recited by the claims, and the terms recited in the claims.

### BRIEF DESCRIPTION OF THE FIGURES

FIG. 1 compares (i) the cumulative sildenafil citrate ODT dissolution as a function of time from formulations G1 (gelatin), X1 (xanthan gum), A1 (AEROSIL200) and C1 (N-CMC) to (ii) sildenafil citrate plain powder and (iii) the market product VIAGRA, according to some embodiments.

FIG. 2 shows the dissolution profiles of sildenafil citrate from ODTs containing 2% gelatin as a matrix former (G1) and different solubilizers, according to some embodiments.

FIG. 3 compares the percentage of sildenafil citrate dissolved from the X1 ODT to ODTs containing 2% xanthan gum as a matrix former and PEG400, PEG6000, and PVPK30 as a solubilizer (X2, X3, and X4), according to some embodiments.

FIG. 4 compares the percentage of sildenafil citrate dissolved from the A1 ODT to ODTs containing 2% AEROSIL200 as a matrix former and PEG400 and PEG6000 as a solubilizer (A2 and A3), according to some embodiments.

FIG. 5 compares the percentage of sildenafil citrate dissolved from the C1 ODT to ODTs containing shows the percent sildenafil citrate dissolved from the ODTs containing 2% Na-CMC as a matrix former and PEG400 and PEG6000 as a solubilizer (C2 and C3), according to some embodiments.

FIG. 6 illustrates dissolution profiles of sildenafil citrate from (i) an ODT with an agent mixture of sildenafil citrate and caffeine (F1) and (ii) the (G5) ODT, according to some embodiments.

FIG. 7 shows the dissolution profiles of caffeine from the F1 ODT, according to some embodiments.

FIG. 8 shows the mean plasma concentration versus time curves of sildenafil citrate following administration of the G5 and F1 ODTs as compared to the commercial oral tablets of VIAGRA administered to the human volunteers, according to some embodiments.

### DETAILED DESCRIPTION

Pharmaceutical compositions and formulations are provided herein comprising a phosphodiesterase type 5 inhibitor, such as sildenafil citrate; and, an adenosine receptor (A1, A2A, A2B, and A3 receptors) antagonist, such as caffeine, for (i) treating erectile dysfunction while (ii) inhibiting the lower of the blood pressure, (iii) increasing the bioavailability of the compositions, and (iv) reducing the Tmax in the subject. Methods of making and administering oral disintegrating tablets are also provided.

In some embodiments, the phosphodiesterase type 5 inhibitors can include, for example, sildenafil citrate (VIAGRA), tadalafil (CIALIS), and vardenafil (LEVITRA), each of which are clinically indicated for the treatment of erectile dysfunction. And, in some embodiments, the phosphodiesterase type 5 inhibitors can include, for example, avanafil, iodenafil, mirodenafil, sildenafil, tadalafil, vardenafil, udenafil, zaprinast, T-1032 (Tanabe Seiyaku Co., Saitama, Japan), benzamidenafil, icariin, and pharmaceutically acceptable salts and derivatives thereof.

In some embodiments, the phosphodiesterase type 5 inhibitor is a pyrazolopyrimidinone having the following structure:

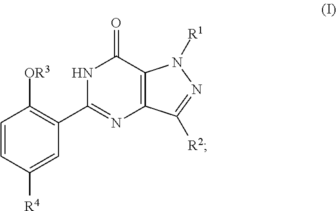

R¹ is H; C₁-C₃ alkyl; C₁-C₃ perfluoroalkyl; or C₃-C₅ cycloalkyl;
R² is H; optionally substituted C₁-C₆ alkyl; C₁-C₃ perfluoroalkyl; or C₃-C₆ cycloalkyl;
R³ is optionally substituted C₁-C₆ alkyl; C₁-C₆ perfluoroalkyl; C₃-C₅ cycloalkyl; C₃-C₆ alkenyl; or C₃-C₆ alkynyl;
R⁴ is optionally substituted C₁-C₄ alkyl, C₂-C₄ alkenyl, C₂-C₄ alkanoyl, (hydroxy)C₂-C₄ alkyl or (C₂-C₃ alkoxy)C₁-C₂ alkyl; CONR₅R₆; CO₂R₇; halo; NR₅R₆, NHSO₂NR₅R₆; NHSO₂R₈; SO₂NR₉R₁₀, or phenyl, pyridyl, pyrimidinyl, imidazolyl, oxazolyl, thiazolyl, thienyl or triazolyl any of which is optionally substituted with methyl;
R⁵ and R⁶ are each independently H or C₁-C₄ alkyl, or together with the nitrogen atom to which they are attached form an optionally substituted pyrrolidinyl, piperidino, morpholino, 4-N(R¹¹)-piperazinyl or imidazolyl group;
R⁷ is H or C₁-C₄ alkyl;
R⁸ is optionally substituted C₁-C₃ alkyl;
R⁹ and R¹⁰ together with the nitrogen atom to which they are attached form an optionally substituted pyrrolidinyl, piperidino, morpholino or 4-N(R¹²)-piperazinyl group;
R¹¹ is H; optionally substituted C₁-C₃ alkyl; (hydroxy)C₂-C₃ alkyl; or C₁-C₄ alkanoyl;
R¹² is H; optionally substituted C₁-C₆ alkyl; CONR¹³R¹⁴; CSNR¹³R¹⁴; or C(NH)NR¹³R¹⁴; and,
R¹³ and R¹⁴ are each independently H; C₁-C₄ alkyl; or substituted C₂-C₄ alkyl;
a pharmaceutically acceptable salt thereof, or a pharmaceutical composition containing either entity, for the manufacture of a medicament for the curative or prophylactic treatment of erectile dysfunction in a male animal, including man.

Unless otherwise indicated, alkyl groups having three or more carbon atoms, alkenyl and alkynyl groups having four or more carbon atoms, alkoxy groups having three carbon atoms and alkanoyl groups having four carbon atoms may be straight chain or branched chain. Halo means fluoro, chloro, bromo or iodo. The compounds of formula (I) may contain one or more asymmetric centers and, thus, they can exist as enantiomers or diastereoisomers. Furthermore, certain compounds of formula (I) which contain alkenyl groups may exist as cis-isomers or trans-isomers. In each instance, the invention includes both mixtures and separate individual isomers.

In some embodiments, the compounds of formula (I) may also exist in tautomeric forms and the invention includes both mixtures and separate individual tautomers. The pharmaceutically acceptable salts of the compounds of formula (I) which contain a basic center are, for example, non-toxic acid addition salts formed with inorganic acids such as hydrochloric, hydrobromic, sulphuric and phosphoric acid, with organo-carboxylic acids, or with organo-sulphonic acids.

In some embodiments, compounds of formula (I) can also provide pharmaceutically acceptable metal salts, in particular non-toxic alkali metal salts, with bases. Examples include the sodium and potassium salts.

In some embodiments of formula (I), R¹ is H, methyl or ethyl; R² is C₁-C₃ alkyl; R³ is C₂-C₃ alkyl or allyl; R⁴ is C₁-C₄ alkyl optionally substituted with OH, NR⁵R⁶, CN, CONR⁵R⁶ or CO₂R⁷; acetyl optionally substituted with NR⁵R⁶; hydroxyethyl optionally substituted with NR⁵R⁶; ethoxymethyl optionally substituted with OH or NR⁵R⁶; CH═CHCN; CH═CHCONR⁵R⁶; CH═CHC0₂R⁷; CONR⁵R⁶; C0₂H; Br; NR⁵R⁶; NHSO₂NR⁵R⁶; NHSO₂R⁸; SO₂NR⁹R¹⁰; or pyridyl or imidazolyl either of which is optionally substituted with methyl; R⁵ and R⁶ are each independently H, methyl or ethyl, or together with the nitrogen atom to which they are attached form a piperidino, morpholino, 4-N(R¹¹)-piperazinyl or imidazolyl group wherein said group is optionally substituted with methyl or OH; R⁷ is H or t-butyl; R⁸ is methyl or CH₂CH₂CH₂NR⁵R⁶; R⁹ and R¹⁰ together with the nitrogen atom to which they are attached form a piperidino or 4-N(R¹²)-piperazinyl group wherein said group is optionally substituted with NR¹³R¹⁴ or CONR¹³R¹⁴; R¹¹ is H, methyl, benzyl, 2-hydroxyethyl or acetyl; R¹² is H, C₁-C₃ alkyl, (hydroxy)C₂-C₃ alkyl, CSNR¹³R¹⁴ or C(NH)NR¹³R¹⁴; and R¹³ and R¹⁴ are each independently H or methyl.

In some embodiments of formula (I), R¹ is methyl or ethyl; R² is C₁-C₃ alkyl; R³ is ethyl, n-propyl or allyl; R⁴ is CH₂NR⁵R⁶, COCH₂NR⁵R⁶, CH(OH)CH₂NR⁵R⁶, CH₂OCH₂CH₃, CH₂OCH₂CH₂OH, CH₂OCH₂CH₂NR⁵R⁶, CH═CHCON(CH₃)₂, CH═CHCO₂R⁷, CONR⁵R⁶, C0₂H, Br, NHSO₂NR⁵R⁶, NHSO₂CH₂CH₂CH₂NR⁵R⁶, SO₂NR⁹R¹⁰, 2-pyridyl, 1-imidazolyl or I-methyl-2-imidazolyl; R⁵ and R⁶ together with the nitrogen atom to which they are attached form a piperidino, 4-hydroxypiperidino, morpholino, 4-N(R¹¹)-piperazinyl or 2-methyl-I-imidazolyl group; R⁷ is H or t-butyl; R⁹ and R¹⁰ together with the nitrogen atom to which they are attached form a 4-carbamoylpiperidino or 4-N(R¹²)-piperazinyl group; R¹¹ is H, methyl, benzyl, 2-hydroxyethyl or acetyl; and R¹² is H, C₁-C₆, alkyl, 2-hydroxyethyl or CSNH₂.

In some embodiments of formula (I), R¹ is methyl or ethyl; R² is n-propyl; R³ is ethyl, n-propyl or allyl; R⁴ is COCH₂NR⁵R⁶, CONR⁵R⁶, SO₂NR⁹R¹⁰ or I-methyl-2-imidazolyl; R⁵ and R⁶ together with the nitrogen atom to which they are attached form a morpholino or 4-N(R¹²)-piperazinyl group; R⁹ and R¹⁰ together with the nitrogen atom to which they are attached form a 4-N(R¹²)-piperazinyl group; R^{u} is methyl or acetyl; and R¹² is H, methyl, 2-propyl or 2-hydroxyethyl.

In some embodiments, formula (I) is the following:
5-(2-ethoxy-5-morpholinoacetylphenyl)-I-methyl-3-n-propyl-I,6-dihydro-7H-pyrazolo[4,3-d]pyrimidin-7-one;
5-(5-morpholinoacetyl-2-n-propoxyphenyl)-1-methyl-3-n-propyl-I,6-dihydro-7H-pyrazolo[4,3-d]pyrimidin-7-one;
5-[2-ethoxy-5-(4-methyl-I-piperazinylsulphonyl)-phenyl]-I-methyl-3-n-propyl-I,6-dihydro-7H-pyrazolo[4,3-d]pyrimidin-7-one;
5-[2-allyloxy-5-(4-methyl-I-piperazinylsulphonyl)-phenyl]-I-methyl-3-n-propyl-I,6-dihydro-7H-pyrazolo[4,3-d]pyrimidin-7-one;
5-{2-ethoxy-5-[4-(2-propyl)-1-piperazinyl-sulphonyl]phenyl}-I-methyl-3-n-propyl-I,6-dihydro-7H-pyrazolo[4,3-d]pyrimidin-7-one;
5-{2-ethoxy-5-[4-(2-hydroxyethyl)-1-piperazinyl-sulphonyl]phenyl}-I-methyl-3-n-propyl-I,6-dihydro-7H-pyrazolo[4,3-d]pyrimidin-7-one;
5-{5-[4-(2-hydroxyethyl)-1-piperazinylsulphonyl]-2-n-propoxyphenyl}-I-methyl-3-n-propyl-I,6-dihydro-7H-pyrazolo[4,3-d]pyrimidin-7-one;
5-[2-ethoxy-5-(4-methyl-I-piperazinylcarbonyl)-phenyl]-I-methyl-3-n-propyl-I,6-dihydro-7H-pyrazolo[4,3-d]pyrimidin-7-one; and,
5-[2-ethoxy-5-(I-methyl-2-imidazolyl)phenyl]-1-methyl-3-n-propyl-I,6-dihydro-7H-pyrazolo[4,3-d]pyrimidin-7-one.

U.S. Pat. Nos. 5,250,534 and 6,469,012, and PCT Publication No. WO1994028902, are each hereby incorporated by reference in their entirety.

In some embodiments, the adenosine receptor antagonist is an antagonist for A1, A2A, A2B, and A3 receptors. In some embodiments, the adenosine receptor antagonist is xanthine, caffeine, theobromine, theophylline, paraxanthine, 8-chlorotheophylline, a pharmaceutically acceptable salt or derivative thereof, or a combination thereof. In some embodiments, the adenosine receptor antagonist is theophylline, or 1,3-dimethyl-7H-purine-2,6-dione, and pharmaceutically acceptable salts and derivatives thereof. In some embodiments, the adenosine receptor antagonist is caffeine, or 1,3,7-trimethylpurine-2,6-dione, and pharmaceutically acceptable salts and derivatives thereof. Examples of a caffeine derivatives can include, for example, (E)-8-(3-Chlorostyryl)-1,3,7-trimethylxanthine. As caffeine is a phosphodiesterase inhibitor, it was surprising and unexpected that combining caffeine with a phosphodiesterase type 5 inhibitor provided the desirable results observed and set-forth herein.

The terms “active agent”, “agent”, “bioactive agent”, “composition”, “drug”, “pharmaceutically active agent”, and “pharmaceutical agent” can be used interchangeably in some embodiments. Each of these terms can be used to refer to any active agent or combination of agents set-forth herein.

The term “bioavailability” can be used to refer to the fraction, or percentage, of an active agent that reaches the systemic circulation of a subject. When an active agent, such as a drug, is administered intravenously, the bioavailability is 100%. However, when a medication is administered via other routes (such as orally), its bioavailability can decrease due to incomplete absorption, first-pass metabolism, and the like, and it can also vary from patient to patient due to variations in physiology. In some embodiments, the active agent's bioavailability can be referred to as the rate and extent to which the agent reaches the systemic circulation of a subject. In some embodiments, the term “bioavailability” can be used interchangeably with “absolute bioavailability” which can be referred to as the bioavailability of the active agent in systemic circulation following non-intravenous administration (i.e., after oral, rectal, transdermal, subcutaneous, or sublingual administration), with the bioavailability of the same drug following intravenous administration.

The terms “Cmax” and “Tmax” are understood by those of skill in the art of pharmacology. The term “Cmax”, for example, can be used to refer to the maximum, or peak, serum concentration that an active agent achieves in the body of a subject after the active agent has been administered, prior to administration of a second dose of the active agent. The term “Tmax”, for example, can be used to refer to the time at which the Cmax is observed. After oral administration of an active agent, the Cmax and Tmax are dependent on the extent, and rate of absorption of the active agent in the subject. As such, Cmax and Tmax can be used to compare absorption rates of formulations. A formulation configured for the primary path of absorption to occur buccally or sublingually can be compared to a formulation configured for the primary path of absorption to occur through the gastrointestinal tract.

As such, compositions are provided that comprise a phosphodiesterase type 5 inhibitor (PDE5 inhibitor), or a pharmaceutically acceptable salt thereof, and caffeine, wherein the composition treats erectile dysfunction in a subject while inhibiting a lowering of blood pressure in a subject. In some embodiments, the PDE5 inhibitor is sildenafil citrate.

Pharmaceutical formulations are provided comprising such a composition, wherein the PDE5 inhibitor is sildenafil citrate; and, the pharmaceutical formulation is in the form of an oral disintegrating tablet designed for a primary absorption through buccal or sublingual mucosa. The tablet can have a matrix former, a sugar alcohol, and a collapse protectant; and, the oral disintegrating tablet provides a relative bioavailability value for the sildenafil citrate that is substantially greater than a tablet designed for a primary absorption through gastrointestinal mucosa.

In some embodiments, the matrix former can be selected from the group consisting of gelatin, xanthan gum, Na-carboxymethyl cellulose, and AEROSIL200. In some embodiments, the sugar alcohol can be selected from the group consisting of mannitol, erythritol, sorbitol, trehalose, xylitol, glucose and sucrose. In some embodiments, the collapse protectant is selected from the group consisting of gelatin and glycine.

The pharmaceutical formulations can further comprise a solubilizer selected from the group consisting of polyethylene glycol, polyvinylpyrrolidone, and polysorbate; wherein, the dissolution rate of the sildenafil citrate in the subject being substantially higher than that of a control group receiving the sildenafil citrate without the solubilizer. In some embodiments, the solubilizer is polyethylene glycol 6000. In some embodiments, the solubilizer is polyvinylpyrrolidone K30. And, in some embodiments, the solubilizer is polysorbate 80.

The pharmaceutical formulations can further comprise a disintegrant selected from the group consisting of croscarmellose sodium, crospovidone, and sodium starch glycoate; wherein, the time to a maximum plasma concentration of the sildenafil citrate in the subject being substantially faster than that of a control group receiving the sildenafil citrate through a commercially available oral tablet configured for a primary absorption through gastrointestinal mucosa.

The term “substantially” can be used to refer to a change in amount that is considered by one of skill to be significantly greater, such that (i) a prophylactic or therapeutic result is significantly better; (ii) a dose is significantly lower; (ii) a rate is significantly faster; (iii) a concentration is significantly higher. An improvement is significantly greater, significantly better, significantly faster, or significantly higher when there is a desired change of at least 10% in some embodiments, at least 20% in some embodiments, at least 30% in some embodiments, at least 40% in some embodiments, at least 50% in some embodiments, or at least 60% in some embodiments. Moreover, traditional statistical analyses can be used to determine when a change is a statistically significant change. In some embodiments, a change is statistically significant when the p-value is less than 0.10, less than 0.075, less than 0.05, less than 0.04, less than 0.03, less than 0.02, or less than 0.01.

The pharmaceutical formulations can further comprise (i) a solubilizer selected from the group consisting of polyethylene glycol, polyvinylpyrrolidone, and polysorbate; and, (ii) a disintegrant selected from the group consisting of croscarmellose sodium, crospovidone, and sodium starch glycoate; wherein, the matrix former is selected from the group consisting of gelatin, xanthan gum, Na-carboxymethyl cellulose, and AEROSIL200; the sugar alcohol is selected from the group consisting of mannitol, erythritol, sorbitol, trehalose, xylitol, glucose and sucrose; and, the collapse protectant is selected from the group consisting of gelatin and glycine.

Uses and Methods of Administration

The compositions provided herein can be used to treat erectile dysfunction. The term “subject” and “patient” are used interchangeably and refer to an animal such as a mammal including, but not limited to, non-primates such as, for example, a cow, pig, horse, cat, dog, rat and mouse; and primates such as, for example, a monkey or a human.

Methods of treating erectile dysfunction in a subject are also provided using any of the compositions, formulations, or articles of manufacture taught herein. The methods can inhibit a reduction in blood pressure of the subject while treating the erectile dysfunction. In some embodiments, the time to a maximum plasma concentration (Tmax) of an active agent is substantially faster than that of a control group receiving the active agent through a commercially available oral tablet configured for a primary absorption through gastrointestinal mucosa. And, in some embodiments, the bioavailability of an active agent in the subject is substantially higher than that of a control group receiving the active agent through a commercially available oral tablet configured for a primary absorption through gastrointestinal mucosa.

The term “primary absorption” can be used to refer to a relative amount of absorption of an active agent that is occurring relative to other types of absorption of the active agent into the blood serum of a subject. In some embodiments, for example, a primary route of absorption is responsible for 100% of the absorption of the active agent into the serum of the subject. In some embodiments, for example, a primary route of absorption is responsible for at least 99% of the absorption of the active agent into the serum of the subject. In some embodiments, for example, a primary route of absorption is responsible for at least 98% of the absorption of the active agent into the serum of the subject. In some embodiments, for example, a primary route of absorption is responsible for at least 97% of the absorption of the active agent into the serum of the subject. In some embodiments, for example, a primary route of absorption is responsible for at least 96% of the absorption of the active agent into the serum of the subject. In some embodiments, for example, a primary route of absorption is responsible for at least 95% of the absorption of the active agent into the serum of the subject. In some embodiments, for example, a primary route of absorption is responsible for at least 94%, at least 93%, at least 92%, at least 91%, at least 90%, at least 89%, at least 88%, at least 87%, at least 86%, at least 85%, at least 84%, at least 83%, at least 82%, at least 81%, at least 80%, at least 75%, at least 70%, at least 65%, at least 60%, or at least 55%, of the absorption of the active agent into the serum of the subject. As described herein, an orally disintegrating tablet can be designed such that the primary route of absorption of an active agent, such as sildenafil citrate and/or caffeine, is a pregrastric route, such as buccally or sublingually, for example. One of skill will understand that some of the absorption can occur through a gastrointestinal route due to, for example, the subject swallowing some of the active agent before the pregastric, buccal or sublingal, administration is completed.

Methods of making an oral disintegrating tablet for treating erectile dysfunction through a buccal or sublingual absorption are also provided. The methods can include combining an effective amount of PDE5 inhibitor with an effective amount of caffeine to create an agent mixture; adding a matrix former, a sugar alcohol, and a collapse protectant to the sildenafil citrate and the caffeine; and, forming an oral disintegrating tablet that functions to deliver the agent mixture through a buccal or sublingual absorption. In some embodiments, the PDE5 inhibitor is sildenafil citrate. In some embodiments, the method further includes adding a disintegrant. In some embodiments, the method further includes adding a solubilizer. In some embodiments, the methods further comprise adding a solubilizer selected from the group consisting of polyethylene glycol, polyvinylpyrrolidone, and polysorbate; and, adding a disintegrant selected from the group consisting of croscarmellose sodium, crospovidone, and sodium starch glycoate; wherein, the matrix former is selected from the group consisting of gelatin, xanthan gum, Na-carboxymethyl cellulose, and AEROSIL200; the sugar alcohol is selected from the group consisting of mannitol, erythritol, sorbitol, trehalose, xylitol, glucose and sucrose; and, the collapse protectant is selected from the group consisting of gelatin and glycine.

The compositions provided herein can be administered to a subject using any manner of administration known to one of skill. For example, in some embodiments, a localized administration is used and, in some embodiments a systemic administration is used. In some embodiments, a combination of system and local administration is used. One of skill will appreciate that the therapeutic program selected, the agents administered, the condition of the subject, and the effects desired, can affect the administration schedule and program used.

One of skill understands that the amount of the agents administered can vary according to factors such as, for example, the type of disease, age, sex, and weight of the subject, as well as the method of administration. For example, local and systemic administration can call for substantially different amounts to be effective. Dosage regimens may also be adjusted to optimize a therapeutic response. In some embodiments, a single bolus may be administered; several divided doses may be administered over time; the dose may be proportionally reduced or increased; or, any combination thereof, as indicated by the exigencies of the therapeutic situation and factors known one of skill in the art. It is to be noted that dosage values may vary with the severity of the condition to be alleviated. Dosage regimens may be adjusted over time according to the individual need and the professional judgment of the person administering or supervising the administration of the compositions, and the dosage ranges set forth herein are exemplary only and do not limit the dosage ranges that may be selected by medical practitioners.

The terms “administration” or “administering” refer to a method of incorporating a composition into the cells or tissues of a subject, either in vivo or ex vivo to diagnose, prevent, treat, or ameliorate a symptom of a disease. In one example, a compound can be administered to a subject in vivo parenterally. In another example, a compound can be administered to a subject by combining the compound with cell tissue from the subject ex vivo for purposes that include, but are not limited to, assays for determining utility and efficacy of a composition. When the compound is incorporated in the subject in combination with one or active agents, the terms “administration” or “administering” can include sequential or concurrent incorporation of the compound with the other agents such as, for example, any agent described above. A pharmaceutical composition of the invention is formulated to be compatible with its intended route of administration. Examples of routes of administration include, but are not limited to, parenteral such as, for example, intravenous, intradermal, intramuscular, and subcutaneous injection; oral; inhalation; intranasal; transdermal; transmucosal; and rectal administration.

An “effective amount” of a compound of the invention can be used to describe a therapeutically effective amount or a prophylactically effective amount. An effective amount can also be an amount that ameliorates the symptoms of a disease. A “therapeutically effective amount” refers to an amount that is effective at the dosages and periods of time necessary to achieve a desired therapeutic result and may also refer to an amount of active compound, prodrug or pharmaceutical agent that elicits any biological or medicinal response in a tissue, system, or subject that is sought by a researcher, veterinarian, medical doctor or other clinician that may be part of a treatment plan leading to a desired effect. In some embodiments, the therapeutically effective amount may need to be administered in an amount sufficient to result in amelioration of one or more symptoms of a disorder, prevention of the advancement of a disorder, or regression of a disorder. In some embodiments, for example, a therapeutically effective amount can refer to the amount of an agent that provides a measurable response of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% of a desired action of the composition. The term “treating” refers to the administering one or more therapeutic or prophylactic agents taught herein.

A “prophylactically effective amount” refers to an amount that is effective at the dosages and periods of time necessary to achieve a desired prophylactic result such as, preventing, or inhibiting, an onset of erectile dysfunction. A prophylactically effective amount may be less than, greater than, or equal to a therapeutically effective amount.

The administration can be local or systemic. In some embodiments, the administration can be oral. In other embodiments, the administration can be subcutaneous injection. In other embodiments, the administration can be intravenous injection using a sterile isotonic aqueous buffer. In another embodiment, the administration can include a solubilizing agent and a local anesthetic such as lignocaine to ease discomfort at the site of injection. In other embodiments, the administrations may be parenteral to obtain, for example, ease and uniformity of administration.

The compounds can be administered in dosage units. The term “dosage unit” refers to discrete, predetermined quantities of a compound that can be administered as unitary dosages to a subject. A predetermined quantity of active compound can be selected to produce a desired therapeutic effect and can be administered with a pharmaceutically acceptable carrier. The predetermined quantity in each unit dosage can depend on factors that include, but are not limited to, (a) the unique characteristics of the active compound and the particular therapeutic effect to be achieved, and (b) the limitations inherent in the art of creating and administering such dosage units.

A “pharmaceutically acceptable carrier” is a diluent, adjuvant, excipient, or vehicle with which the composition is administered. A carrier is pharmaceutically acceptable after approval by a state or federal regulatory agency or listing in the U.S. Pharmacopeial Convention or other generally recognized sources for use in subjects.

The pharmaceutical carriers include any and all physiologically compatible solvents, dispersion media, coatings, antibacterial and antifungal agents, isotonic and absorption delaying agents, and the like. Examples of pharmaceutical carriers include, but are not limited to, sterile liquids, such as water, oils and lipids such as, for example, phospholipids and glycolipids. These sterile liquids include, but are not limited to, those derived from petroleum, animal, vegetable or synthetic origin such as, for example, peanut oil, soybean oil, mineral oil, sesame oil, and the like. Water can be a preferred carrier for intravenous administration. Saline solutions, aqueous dextrose and glycerol solutions can also be liquid carriers, particularly for injectable solutions.

Suitable pharmaceutical excipients include, but are not limited to, starch, sugars, inert polymers, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition can also contain minor amounts of wetting agents, emulsifying agents, pH buffering agents, or a combination thereof. The compositions can take the form of solutions, suspensions, emulsion, tablets, pills, capsules, powders, sustained-release formulations and the like. Oral formulations can include standard carriers such as, for example, pharmaceutical grades mannitol, lactose, starch, magnesium stearate, sodium saccharine, cellulose, magnesium carbonate, and the like. See Martin, E. W. Remington's Pharmaceutical Sciences. Supplementary active compounds can also be incorporated into the compositions.

In some embodiments, the carrier is suitable for parenteral administration. In other embodiments, the carrier can be suitable for intravenous, intraperitoneal, intramuscular, sublingual or oral administration. In other embodiments, the pharmaceutically acceptable carrier may comprise pharmaceutically acceptable salts.

Pharmaceutical formulations for parenteral administration may include liposomes. Liposomes and emulsions are delivery vehicles or carriers that are especially useful for hydrophobic drugs. Depending on biological stability of the therapeutic reagent, additional strategies for protein stabilization may be employed. Furthermore, one may administer the drug in a targeted drug delivery system such as, for example, in a liposome coated with target-specific antibody. The liposomes can be designed, for example, to bind to a target protein and be taken up selectively by the cell expressing the target protein.

Therapeutic compositions typically must be sterile and stable under the conditions of manufacture and storage. The composition can be formulated as a solution, microemulsion, liposome, or other ordered structure suitable for a high drug concentration. In some embodiments, the carrier can be a solvent or dispersion medium including, but not limited to, water; ethanol; a polyol such as for example, glycerol, propylene glycol, liquid polyethylene glycol, and the like; and, combinations thereof. The proper fluidity can be maintained in a variety of ways such as, for example, using a coating such as lecithin, maintaining a required particle size in dispersions, and using surfactants.

In some embodiments, isotonic agents can be used such as, for example, sugars; polyalcohols that include, but are not limited to, mannitol, sorbitol, glycerol, and combinations thereof; and sodium chloride. Sustained absorption characteristics can be introduced into the compositions by including agents that delay absorption such as, for example, monostearate salts, gelatin, and slow release polymers. Carriers can be used to protect active compounds against rapid release, and such carriers include, but are not limited to, controlled release formulations in implants and microencapsulated delivery systems. Biodegradable and biocompatible polymers can be used such as, for example, ethylene vinyl acetate, polyanhydrides, polyglycolic acid, collagen, polyorthoesters, polylactic acid, polycaprolactone, polyglycolic copolymer (PLG), and the like. Such formulations can generally be prepared using methods known to one of skill in the art.

The compounds may be administered as suspensions such as, for example, oily suspensions for injection. Lipophilic solvents or vehicles include, but are not limited to, fatty oils such as, for example, sesame oil; synthetic fatty acid esters, such as ethyl oleate or triglycerides; and liposomes. Suspensions that can be used for injection may also contain substances that increase the viscosity of the suspension such as, for example, sodium carboxymethyl cellulose, sorbitol, or dextran. Optionally, a suspension may contain stabilizers or agents that increase the solubility of the compounds and allow for preparation of highly concentrated solutions.

In one embodiment, a sterile and injectable solution can be prepared by incorporating an effective amount of an active compound in a solvent with any one or any combination of desired additional ingredients described above, filtering, and then sterilizing the solution. In another embodiment, dispersions can be prepared by incorporating an active compound into a sterile vehicle containing a dispersion medium and any one or any combination of desired additional ingredients described above. Sterile powders can be prepared for use in sterile and injectable solutions by vacuum drying, freeze-drying, or a combination thereof, to yield a powder that can be comprised of the active ingredient and any desired additional ingredients. Moreover, the additional ingredients can be from a separately prepared sterile and filtered solution. In another embodiment, the extract may be prepared in combination with one or more additional compounds that enhance the solubility of the extract.

In some embodiments, a therapeutically or prophylactically effective amount of a composition may range in blood serum concentration of an active agent from about 0.001 nM to about 0.10 M; from about 0.001 nM to about 0.5 M; from about 0.01 nM to about 150 nM; from about 0.01 nM to about 500 μM; from about 0.01 nM to about 1000 nM, 0.001 μM to about 0.10 M; from about 0.001 μM to about 0.5 M; from about 0.01 μM to about 150 μM; from about 0.01 μM to about 500 μM; from about 0.01 μM to about 1000 nM, or any range therein. In some embodiments, the compositions may be administered in an amount ranging from about 0.001 mg/kg to about 500 mg/kg; from about 0.005 mg/kg to about 400 mg/kg; from about 0.01 mg/kg to about 300 mg/kg; from about 0.01 mg/kg to about 250 mg/kg; from about 0.1 mg/kg to about 200 mg/kg; from about 0.2 mg/kg to about 150 mg/kg; from about 0.4 mg/kg to about 120 mg/kg; from about 0.15 mg/kg to about 100 mg/kg, from about 0.15 mg/kg to about 50 mg/kg, from about 0.5 mg/kg to about 10 mg/kg, or any range therein, wherein a human subject is assumed to average about 70 kg.

In some embodiments, a dosage form herein includes a range of about 10 mg to about 100 mg of sildenafil citrate given approximately 1 hour before sexual activity in a patient averaging 70 kg in body weight. As such, the dosage of the phosphodiesterase type 5 inhibitor can range from about 100 μg/kg to about 1 mg/kg, in some embodiments. In some embodiments, the dosage of phosphodiesterase type 5 inhibitor can range from about 10 μg/kg to about 10 mg/kg, in some embodiments. However, the dosage may be taken anywhere in the range of about 10 minutes to about 4 hours before sexual activity. In some embodiments, the dosage may be taken about 5 minutes, about 10 minutes, about 15 minutes, about 20 minutes, about 30 minutes, about 40 minutes, about 50 minutes, about 60 minutes, about 70 minutes, about 80 minutes, or any amount therein in increments of 5 minutes. In some embodiments, the maximum dosing frequency is once per day.

In some embodiments, the active agent combination (phosphodiesterase type 5 inhibitor and adenosine receptor antagonist) comprises up to a range of about 50% to about 90% of the tablet by weight. In some embodiments, the active agent combination (phosphodiesterase type 5 inhibitor and adenosine receptor antagonist) comprises up to 50%, 60%, 70%, 80%, 90%, or any range therein in increments of 1%, of the tablet by weight. In some embodiments, the active agent combination (phosphodiesterase type 5 inhibitor and adenosine receptor antagonist) comprises up to 60% of the tablet by weight. The remaining components can be, for example, up to 2.2% matrix former, up to 22% sugar alcohol, up to 2.2% disintegrant, up to 1.1% protectant, and up to 2.2% solubilizer. In some embodiments, the active agent combination (phosphodiesterase type 5 inhibitor and adenosine receptor antagonist) comprises up to 70% of the tablet by weight. The remaining components can be, for example, up to 2% matrix former, up to 20% sugar alcohol, up to 2% disintegrant, up to 1% protectant, and up to 2% solubilizer. In some embodiments, the active agent combination (phosphodiesterase type 5 inhibitor and adenosine receptor antagonist) comprises up to a 80% of the tablet by weight. The remaining components can be, for example, up to 1.8% matrix former, up to 18% sugar alcohol, up to 1.8% disintegrant, up to 0.8% protectant, and up to 1.8% solubilizer. In some embodiments, the active agent combination (phosphodiesterase type 5 inhibitor and adenosine receptor antagonist) comprises up to 90% of the tablet by weight. The remaining components can be, for example, up to 1.6% matrix former, up to 16% sugar alcohol, up to 1.6% disintegrant, up to 0.6% protectant, and up to 1.6% solubilizer. As described herein, the phosphodiesterase type 5 inhibitor can be sildenafil citrate, or a pharmaceutically acceptable derivative or salt thereof; and, the adenosine receptor antagonist is caffeine, or a pharmaceutically acceptable derivative or salt thereof.

The weight ratio of phosphodiesterase type 5 inhibitor to adenosine receptor antagonist can range from about 1:10 to about 10:1, from about 1:5 to about 5:1, from about 1:3 to about 3:1, from about 1:2 to about 2:1, from about 1.9:1 to about 1:1.9, from about 1.8:1 to about 1:1.8, from about 1.7:1 to about 1:1.7, from about 1.6:1 to about 1:1.6, from about 1.5:1 to about 1:1.5, from about 1.4:1 to about 1:1.4, from about 1.3:1 to about 1:1.3, from about 1.2:1 to about 1:1.2, from about 1.1:1 to about 1:1.1, or about 1:1. As described herein, the phosphodiesterase type 5 inhibitor can be sildenafil citrate, or a pharmaceutically acceptable derivative or salt thereof; and, the adenosine receptor antagonist is caffeine, or a pharmaceutically acceptable derivative or salt thereof.

In some embodiments, the phosphodiesterase type 5 inhibitor can be administered in an amount ranging from about 20 mg to about 200 mg, from about 25 mg to about 175 mg, from about 30 mg to about 150 mg, from about 40 mg to about 125 mg, or any range therein in increments of 5 mg. In some embodiments, the adenosine receptor antagonist can be administered in an amount ranging from about 20 mg to about 200 mg, from about 25 mg to about 175 mg, from about 30 mg to about 150 mg, from about 40 mg to about 125 mg, or any range therein in increments of 5 mg. As described herein, the phosphodiesterase type 5 inhibitor can be sildenafil citrate, or a pharmaceutically acceptable derivative or salt thereof; and, the adenosine receptor antagonist is caffeine, or a pharmaceutically acceptable derivative or salt thereof.

In some embodiments, the compounds can be administered by inhalation through an aerosol spray or a nebulizer that may include a suitable propellant such as, for example, dichlorodifluoromethane, trichlorofluoromethane, dichlorotetrafluoroethane, carbon dioxide, or a combination thereof. In one example, a dosage unit for a pressurized aerosol may be delivered through a metering valve. In another embodiment, capsules and cartridges of gelatin, for example, may be used in an inhaler and can be formulated to contain a powderized mix of the compound with a suitable powder base such as, for example, starch or lactose.

Also provided are sustained release formulations for the administration of one or more agents. In some embodiments, the sustained release formulations can reduce the dosage and/or frequency of the administrations of such agents to a subject.

The compositions can be administered as a pharmaceutical formulation by injection. In some embodiments, the formulation can comprise the extract in combination with an aqueous injectable excipient. Examples of suitable aqueous injectable excipients are well known to persons of ordinary skill in the art, and they, and the methods of formulating the formulations, may be found in such standard references as Alfonso A R: Remington's Pharmaceutical Sciences, 17th ed., Mack Publishing Company, Easton Pa., 1985. Suitable aqueous injectable excipients include water, aqueous saline solution, aqueous dextrose solution, and the like, optionally containing dissolution enhancers for the acid-modified arabinogalactan protein composition, such as solution of mannitol or other sugars, or a solution of glycine or other amino acids.

Typically, a composition taught herein can be administered by subcutaneously, intramuscularly, intraperitoneally, or intravenously, injecting. A localized administration can, in some embodiments, include direct injection of an agent into the region of the tissue to be treated. In some embodiments, intravenous administration is used, and it can be continuous intravenous infusion over a period of a few minutes to an hour or more, such as around fifteen minutes. The amount administered may vary widely depending on the type of formulation, size of a unit dosage, kind of excipients, and other factors well known to those of ordinary skill in the art. The formulation may comprise, for example, from about 0.0001% to about 10% (w/w), from about 0.01% to about 1%, from about 0.1% to about 0.8%, or any range therein, with the remainder comprising the excipient or excipients.

In some embodiments, the composition can be administered in conjunction with at least one other therapeutic agent. The amounts of the agents needed can be reduced, even substantially, such that the amount of the agent or agents required is reduced to the extent that a significant response is observed from the subject. A significant response can include, but is not limited to, any known side effects including a reduction or elimination of nausea, a visible increase in tolerance, a faster response to the treatment, a more selective response to the treatment, or a combination thereof. In some embodiments, the side effects treated by the administration of an agent, such as the at least one other agent include, for example, facial flushing, headaches, stomach pain, nasal congestion, nausea, diarrhea, and an inability to differentiate between the colors green and blue.

Orally Disintegrating Tablets

The term “orally disintegrating tablet” can be referred to interchangeably with “ODT”, “orodisperse tablet”, “mouth dissolving tablet”, “rapidly disintegrating tablet”, “fast melt tablet”, and “quick dissolve system”, in some embodiments. The compositions taught herein can be administered in such orally disintegrating tablet dosage forms designed to disintegrate rapidly on contact with saliva and enable oral administration without water or chewing. In some embodiments, the orally disintegrating tablets should disintegrate in the mouth of a subject within a range of 1 second to 5 minutes, within a range of 3 seconds to 3 minutes, within a range of 3 seconds to 2 minutes, within a range of 3 seconds to 1 minute, within a range of 1 second to 30 seconds, within a range of 2 seconds to 25 seconds, within a range of 2 seconds to 20 seconds, within a range of 2 seconds to 15 seconds, within a range of 2 seconds to 15 seconds, within a range of 3 seconds to 20 seconds, or any range therein in increments of 1 second. In some embodiments the orally disintegrating tablets should disintegrate in the mouth no slower than 2 seconds, 3 seconds, 5 seconds, 10 seconds, 15 seconds, 20 seconds, 25 seconds, or any time therein in increments of 1 second.

The orally disintegrating tablets can be configured to improve the drug dissolution, speed up the onset of the clinical effect of the drug when compared to conventional tablets configured for gastrointestinal absorption, avoid the first pass hepatic metabolism of conventional tablets configured for gastrointestinal absorption that reduces the dose, and increase the bioavailability of the drug. In some embodiments, the orally disintegrating tablets are configured for the primary absorption of the drug through pregastric absorption through the oromucosal tissues such as buccally and sublingually, including the oral cavity, pharynx, and esophagus. As such, it should be appreciated that in some embodiments, the dosage of the orally disintegrating tablet dosage forms can be substantially less than the dosage of the conventional tablets designed for gastrointestinal absorption.

One of skill will appreciate that the orally disintegrating tablets can be formed in a variety of ways including, but not limited to, the following:

Direct compression—conventional compression equipment can be used with commonly available excipients. In some embodiments, superdisintegrants can be used, for example, at a concentration ranging from about 2% to about 5%.

Lyophilization or Freeze-drying—freeze-drying or lyophilization can be used to create a desired morphology to the structure that can speed-up disintegration of the tablet and dissolution of the drug. This method includes (i) freezing to bring the material below its eutectic zone, (ii) sublimation or primary drying to reduce moisture to around 4% wt/wt of the dry product, and (iii) desorption or secondary drying to reduce bound moisture to the required final volume. In some embodiments, a sugar alcohol can be added to enhance the characteristics of the dosage form. In some embodiments, mannitol can be added as a cryoprotectant to induce crystallinity, improve rigidity to an amorphous lyophilized or freeze-dried structure, prevent collapse of the structure, and mask an otherwise bitter taste.

Spray drying—this process can be used to quickly remove solvents and produce a highly porous fine powder that dissolves rapidly. A particulate support matrix is prepared by spray drying an aqueous composition containing support matrix and other components, then mixed with active ingredients and compressing them into tablets. The formulations can be incorporated by hydrolyzed and non-hydrolyzed gelatins as supporting agents, mannitol as bulking agent, sodium starch glycolate or crosscarmellose sodium as a disintegrating agent and, in some embodiments, an acidic material (e.g. citric acid) and/or alkali material (e.g. sodium bicarbonate) to enhance disintegration and dissolution. A tablet can then be compressed from the spray-dried powder. Such formulations can use a hydrolyzed/non hydrolyzed gelatin as a supporting agent for the matrix, mannitol as a bulking agent, and sodium starch glycolate or croscarmellose sodium as a disintegrating agent, in some embodiments.

Molding—molded tablets can be prepared from water-soluble sugars. A powdered blend containing drug and excipients like binding agents, e.g., sucrose, acacia, polyvinyl pyrrolidone, etc., can be pushed through a very fine screen to ensure rapid dissolution, moistened with a hydro-alcoholic solvent, and molded into tablets under a pressure that is lower than the pressures typically used for conventional compressed tablets. The solvent is later removed by air-drying to create a porous structure that enhances dissolution. Water soluble ingredients can be used to facilitate absorption through mucosal lining of mouth, for example, thus increasing bioavailability and decreasing first-pass metabolism of drugs by bypassing gastrointestinal absorption. Soluble ingredients, such as saccharides, improve disintegration of tablets and have a low mechanical strength, facilitating erosion and dissolution of the tablet. Compression molding, beat molding, and molding by vacuum without lyophilization can be used.

Other methods—other formation methods include phase transition, melt granulation, sublimation, mass extrusion, and forming thin films.

A fast-disintegrating tablets may have many numerous shapes, such as dish-like, ellipsoid, rods, granules, blocks, cubes with rounded edges, or any other shape suitable for pharmaceutical administration. The compositions of an orally disintegrating tablet include, generally speaking, an active agent and a variety of excipients that include a matrix former, a sugar alcohol, and a collapse protectant. In some embodiments, the orally disintegrating tablet can also include a solubilizer. In some embodiments, the orally disintegrating tablet can include a disintegrant. And, in some embodiments, the orally disintegrating tablet includes both the solubilizer and the disintegrant.

Excipients can be selected from the group consisting of pregelatinized starches, polyvinylpyrrolidone, methylcellulose, microcrystalline cellulose, sucrose, lactose, dextrose, sorbitol, mannitol, lactitol, xylitol, modified calcium salt, granulated corn starch, modified rice starch, compressible sugar, dextrate, dicalcium phosphate, hydroxypropylcellulose, methylcellulose, hydroxypropylmethylcellulose, polyethylene glycol, amylose, anhydrous calcium hydrogen phosphate, calcium sulphate, maltose, tribasic calcium phosphate, dibasic calcium phosphate, low-crystallinity powdered cellulose, silicified microcrystalline cellulose, chitin, chitosan hydrochloride, copovidone, croscarmellose sodium, dextrose, anhydrous lactose, anhydrous alpha lactose, anhydrous beta lactose, agglomerated lactose, spray-dried lactose, maltodextrin, co-processed anhydrous lactose-anhydrous lactitol, co-processed calcium sulphate-microcrystalline cellulose, fructose, co-processed lactose-cellulose, co-processed lactose-starch, co-processed lactose-povidone, coprecipitated sucrose-maltodextrin, carbohydrates such as erythritol, isomalt, lacitol, maltitol, starch hydrolysate, polydextrose, glucose, and mixtures thereof. It is to be appreciated that, in some embodiments, any one or more of the excipients can be excluded from a desired mixture or set of mixtures possible.

As such, a dosage form provided herein can include one or more pharmaceutically acceptable excipients, carriers, or diluents. For example, a dosage form can include a surfactant, a diluent, a sweetener, a disintegrant, a binder, a lubricant, a glidant, a colorant, a flavor, a stabilizing agent, or a mixture thereof.

In some embodiments, excipients can include, but are not limited to, calcium sulfate, starch, mannitol, kaolin, sorbitol, xylitol, sodium chloride, sodium bicarbonate, citric acid, powdered cellulose derivatives, microcrystalline cellulose, pullulan, silicified microcrystalline cellulose, ammonium bicarbonate, carrageenan, carbohydrates, magnesium carbonate, tribasic calcium phosphate, calcium sulfate, magnesium oxide, poloxamer, gums, hydroxypropyl methylcellulose, gelatin, or a mixture thereof.

In some embodiments, diluents can include, but are not limited to, mannitol, sorbitol, xylitol, microcrystalline cellulose, silicified microcrystalline cellulose, hydroxypropyl methylcellulose, hydroxypropyl cellulose, pullulan, a carbohydrate, or a mixture thereof.

In some embodiments, glidants can include, but are not limited to, silicon dioxide, colloidal silicon dioxide, calcium silicate, magnesium silicate, magnesium trisilicate, talc, starch, or a mixture thereof.

In some embodiments, binders can include, but are not limited to, sodium alginate, cellulose, methylcellulose, ethylcellulose, hydroxypropyl cellulose, hydroxypropyl methylcellulose, sodium carboxymethyl cellulose, polypropylpyrrolidone, polyvinylprrolidone, gelatin, polyethylene glycol, starch, pre-gelatinized starch, sugars, trehalose, glucose, tragacanth, sorbitol, acacia, alginates, carrageenan, xanthan gum, locust bean gum and gum arabic, waxes, polyacrylamide, or a mixture thereof.

In some embodiments, lubricants can include, but are not limited to, calcium stearate, glyceryl monostearate, glyceryl behenate, glyceryl palmitostearate, hydrogenated vegetable oil, light mineral oil, magnesium stearate, mineral oil, polyethylene glycol, poloxamer, sodium benzoate, sodium lauryl sulfate, sodium stearyl fumarate, stearic acid, talc, zinc stearate, or a mixture thereof.

In some embodiments disintegrants can include, but are not limited to, sodium starch glycolate, sodium carboxymethyl cellulose, calcium carboxymethyl cellulose, croscarmellose sodium, crospovidone, chitosan, agar, alginic acid, calcium alginate, methyl cellulose, microcrystalline cellulose, powdered cellulose, lower alkylsubstituted hydroxypropyl cellulose, hydroxylpropyl starch, low-substituted hydroxypropylcellulose, polacrilin potassium, starch, pregelatinized starch, sodium alginate, magnesium aluminum silicate, polacrilin potassium, povidone, sodium starch glycolate, or a mixtures thereof.

In some embodiments, flavors can include, but are not limited to, cinnamon oil, essence of apple, essence of pear, essence of peach, essence of grape, essence of strawberry, essence of raspberry, essence of cherry, essence of plum, essence of pineapple, essence of apricot, oil of wintergreen, peppermint oils, clove oil, bay oil, anise oil, eucalyptus, thyme oil, cedar leaf oil, oil of nutmeg, oil of sage, oil of bitter almonds, cassia oil, citrus oils such as lemon, orange, grape, lime and grapefruit, gurana, marshmallow, berries, vanilla, benzaldehyde, aldehyde C-8, licorice, raspberry, aldehyde C-9, aldehyde C-12, tolyl aldehyde, bubble gum, sarsaparilla compound, miracle berry, aromatic elixir, extracts of gurana, glycyrrhiza elixir, or a mixture thereof.

In some embodiments, sweeteners can include, but are not limited to, corn syrup, dextrose, invert sugar, fructose, saccharin, aspartame, acesulfame-K, Stevia rebaudiana, sucralose, sorbitol, mannitol, zylitol, or a mixture thereof.

Other ingredients such as colorants and titanium dioxide can also be used in the dosage forms taught herein, as well as sugar-based excipients, super-disintegrating agents, effervescent agents and further suitable excipients. Moreover, the dosage forms provided herein can include a carbohydrate-based fast dissolving dosage form. Co-processed carbohydrates can be used in embodiments. The orally disintegrating dosage forms provided herein typically dissolve or disperse rapidly when in contact with saliva.

Articles of Manufacture

The present invention provides for articles of manufacture that encompass finished, packaged and labelled pharmaceutical products. The articles of manufacture include the appropriate unit dosage form in an appropriate vessel or container such as, for example, a glass vial or other container that is hermetically sealed. In most embodiments, the dosage form will be an orally disintegrating tablet. Alternatively, the unit dosage form may be a solid suitable for conventional oral, transdermal, topical or mucosal delivery.

As with any pharmaceutical product, the packaging material and container are designed to protect the stability of the product during storage and shipment. In addition, the articles of manufacture can include instructions for use or other information material that can advise the user such as, for example, a physician, technician or patient, regarding how to properly administer the composition as a prophylactic, therapeutic, or ameliorative treatment. In some embodiments, instructions can indicate or suggest a dosing regimen that includes, but is not limited to, actual doses and monitoring procedures.

In other embodiments, the instructions can include informational material indicating that the administering of the compositions can result in adverse reactions including but not limited to allergic reactions such as, for example, anaphylaxis. The informational material can indicate that allergic reactions may exhibit only as mild pruritic rashes or may be severe and include erythroderma, vasculitis, anaphylaxis, Steven-Johnson syndrome, and the like. The informational material should indicate that anaphylaxis can be fatal and may occur when any foreign protein is introduced into the body. The informational material should indicate that these allergic reactions can manifest themselves as urticaria or a rash and develop into lethal systemic reactions and can occur soon after exposure such as, for example, within 10 minutes. The informational material can further indicate that an allergic reaction may cause a subject to experience paresthesia, hypotension, laryngeal edema, mental status changes, facial or pharyngeal angioedema, airway obstruction, bronchospasm, urticaria and pruritus, serum sickness, arthritis, allergic nephritis, glomerulonephritis, temporal arthritis, eosinophilia, or a combination thereof.

In some embodiments, the articles of manufacture can comprise one or more packaging materials such as, for example, a box, bottle, tube, vial, container, sprayer, envelope, and the like; and at least one unit dosage form of an agent taught herein within the packaging material. In other embodiments, the articles of manufacture may also include instructions for using the composition as a prophylactic or therapeutic treatment.

Without intending to be limited to any theory or mechanism of action, the following examples are provided to further illustrate the teachings presented herein. It should be appreciated that there are several variations contemplated within the skill in the art, and that the examples are not intended to be construed as providing limitations to the claims.

In the following examples, a variety of sildenafil citrate oral disintegrating tablets (ODTs) were prepared using a freeze drying technique (lyophilization). Various excipients were tested for their ability at improving oral disintegration, dissolution and bioavailability of the active agent in the ODTs to treat erectile dysfunction. Caffeine was added to the best selected formula of the ODTs to inhibit the expected blood pressure from the administration of the sildenafil citrate. The physicopharmaceutical and solid-state properties, as well as dissolution behavior, of the various ODTs were evaluated. Moreover, the bioavailability of the sildenafil citrate obtained in human volunteers from administration of the various ODTs was compared to that of the market product oral tablet (VIAGRA). The effect of adding caffeine to the best selected ODT on the blood pressure of the human volunteers was also monitored. It was found that using gelatin as a matrix former with polysorbate80 as a solubilizer enhanced the dissolution rate and extent of the sildenafil citrate, wherein 100% of sildenafil citrate was dissolved after 7 minutes. An in vivo study showed that the AUC₀₋₁₂ of the lyophilized ODTs was higher than that of VIAGRA, wherein the ODTs provided a surprisingly high relative bioavailability values of 122% (sildenafil citrate alone) and 125% (an agent mixture of sildenafil citrate and caffeine), respectively, when compared to VIAGRA. Also surprisingly, the addition of caffeine to the best selected ODTs either maintained blood pressure, or inhibited a severe decrease in the blood pressure, when compared to the best selected ODTs and VIAGRA. As such, the examples show that an agent mixture of sildenafil citrate and caffeine treats erectile dysfunction while inhibiting the side-effect expected, associated blood pressure drop. Even more surprisingly, the ODTs taught herein provide a dosage form that bypasses the need for a primary absorption in the gastrointestinal tract, such that the ODTs (i) increase Cmax, (ii) increase Tmax, and (iii) increase bioavailability of the sildenafil citrate while both (iv) treating erectile dysfunction and (v) inhibiting the expected, associated side-effect of a blood pressure drop.

### EXAMPLE 1

### Making an Orally Disintegrating Tablets (ODTs) for Absorption of Sildenafil Citrate Through Buccal or Sublingual Mucosa

This example teaches how orally disintegrating tablets have been prepared, according to some embodiments.

### Materials

The following materials were used in the studies provided herein: sildenafil citrate (Copad Pharma, Egypt); caffeine (Egyptian International Pharmaceuticals Industries Co. (EIPICO)); mannitol (Roquette Pharma, France); gelatin, glycine, TWEEN80, Sodium chloride and Potassium chloride (Adwic, El-Nasr Pharmaceutical Chemicals Co., Egypt); sodium carboxymethyl cellulose (Na-CMC), AEROSIL200 (hydrophilic fumed silica with a specific surface area of 200 m²/g), aspartame and croscarmellose sodium (DELTA PHARMA); xanthan gum (MP Biomedicals, Inc., France), polyethylene glycol (PEG 400 and PEG 6000), polyvinylpyrrolidone (PVP K30), and (β-cyclodextrins) (Fluka AG, Buchs. Switzerland); disodium hydrogen phosphate, potassium dihydrogen phosphate, and methanol (Karl Fischer grade); HYDRANAL Composite 5 reagent (Riedel-de Haën, Sigma-Aldrich. GmbH. Germany); omeprazole (Cobad pharma); human plasma (Vacsera Blood Bank); methanol and acetonitrile HPLC grade (Scharlau, Spain); and, ammonium formate (Sigma-Aldrich, Germany).

### Methods

I. Preparation of Sildenafil Citrate Orally Disintegrating Tablets (ODTs):

Sildenafil citrate ODTs were prepared using four matrix formers, along with some other excipients, and a collapse protectant. The four matrix formers were gelatin (2% w/w), xanthan gum (2% w/w), Na-carboxymethyl cellulose (2% w/w), and AEROSIL200 (2% w/w). Other excipients used were croscarmellose sodium as a super disintegrant, mannitol as a filler, glycine as a collapse protectant and aspartame as a sweetener (sildenafil citrate has a bitter flavor). An accurately weighed amount of sildenafil citrate powder was dispersed in an aqueous solution containing the matrix former and other excipients using magnetic stirrer (Thermolyn Corporation, Dubuque, Lowa, USA) to result in a dose of 70 mg of sildenafil citrate per one milliliter of the resulting suspension.

One milliliter of the suspension was then poured into each of the pockets of a polyvinyl chloride blister pack, creating a dose of 70 mg sildenafil citrate per tablet. The tablet blister pack was then transferred to a freezer at −22° C. and kept in the freezer for 24 hours. The frozen tablets were then placed in a lyophilizer for 24 hours using a Novalvphe-NL 500 Freeze Dryer with a condenser temperature of −45° C. and a pressure of 0.07 mbar. Different formulae were also prepared after adding different solubilizers to the previously described aqueous solution, such as PEG (polyethylene glycol) 400, PEG 6000, PVP (polyvinylpyrrolidone) K30 and TWEEN80 (polysorbate 80). Complexes of sildenafil citrate with (β-cyclodextrin) (1:1 molar ratio) were prepared using the freeze drying method, and these complexes were also tested and compared to the variety of other ODTs. The lyophilized tablets were kept in tightly closed containers in desiccators over anhydrous calcium chloride (29% relative humidity) at room temperature until tested. Sildenafil citrate/caffeine ODTs were prepared by adding 70 mg of caffeine to the best selected ODT and compared to the best selected ODT having sildenafil citrate as the only active agent. Compositions of all tablet formulae are presented in Table 1.

TABLE 1

| | | | | | | Super disintegrant | Collapse | | | |
|---|---|---|---|---|---|---|---|---|---|---|
| | Matrix former | | | | Filler | (mg) | protectant | Sweetener | | |
| For- | (mg) | | | | (mg) | Croscarmellose | (mg) | (mg) | Solubilizer | Caffeine |
| mula | G* | X* | A* | C* | Mannitol | sodium | Glycine | Aspartame | (mg) | (mg) |
| G1 | 4 | — | — | — | 115.2 | 4 | 1.80 | 5 | — | — |
| G2 | 4 | — | — | — | 111.2 | 4 | 1.80 | 5 | 4 mg PEG400 | — |
| G3 | 4 | — | — | — | 111.2 | 4 | 1.80 | 5 | 4 mg PEG6000 | — |
| G4 | 4 | — | — | — | 111.2 | 4 | 1.80 | 5 | 4 mg PVPK30 | — |
| G5 | 4 | — | — | — | 111.2 | 4 | 1.80 | 5 | 4 mg tween80 | — |
| G6 | 4 | — | — | — | 45.20 | 4 | 1.80 | 5 | 70 mg β-CD** | — |
| X1 | — | 4 | — | — | 115.2 | 4 | 1.80 | 5 | — | — |
| X2 | — | 4 | — | — | 111.2 | 4 | 1.80 | 5 | 4 mg PEG400 | — |
| X3 | — | 4 | — | — | 111.2 | 4 | 1.80 | 5 | 4 mg PEG6000 | — |
| X4 | — | 4 | — | — | 111.2 | 4 | 1.80 | 5 | 4 mg PVPK30 | — |
| X5 | — | 4 | — | — | 111.2 | 4 | 1.80 | 5 | 4 mg tween80 | — |
| X6 | — | 4 | — | — | 45.20 | 4 | 1.80 | 5 | 1:1 β-CD** | — |
| A1 | — | — | 4 | — | 115.2 | 4 | 1.80 | 5 | — | — |
| A2 | — | — | 4 | — | 111.2 | 4 | 1.80 | 5 | 4 mg PEG400 | — |
| A3 | — | — | 4 | — | 111.2 | 4 | 1.80 | 5 | 4 mg PEG6000 | — |
| A4 | — | — | 4 | — | 111.2 | 4 | 1.80 | 5 | 4 mg PVPK30 | — |
| A5 | — | — | 4 | — | 111.2 | 4 | 1.80 | 5 | 4 mg tween80 | — |
| A6 | — | — | 4 | — | 45.20 | 4 | 1.80 | 5 | 1:1 β-CD** | — |
| C1 | — | — | — | 4 | 115.2 | 4 | 1.80 | 5 | — | — |
| C2 | — | — | — | 4 | 111.2 | 4 | 1.80 | 5 | 4 mg PEG400 | — |
| C3 | — | — | — | 4 | 111.2 | 4 | 1.80 | 5 | 4 mg PEG6000 | — |
| C4 | — | — | — | 4 | 111.2 | 4 | 1.80 | 5 | 4 mg PVPK30 | — |
| C5 | — | — | — | 4 | 111.2 | 4 | 1.80 | 5 | 4 mg tween80 | — |
| C6 | — | — | — | 4 | 45.20 | 4 | 1.80 | 5 | 70 mg β-CD** | — |
| F1 | 4 | — | — | — | 41.2 | 4 | 1.80 | 5 | 4 mg tween80 | 70 |

All formulations contain 70 mg sildenafil citrate
*different matrix former (G = gelatin; X = xanthan gum; A = AEROSIL200; C = sodium carboxymethyl cellulose)
**β-CD = β-cyclodextrin

II. Preparation of the Physical Mixtures:

Sildenafil citrate powder was uniformly mixed with the excipients used in the formulae in the same percentage used in the lyophilized tablets using a mortar and pestle. The physical mixture was prepared for comparisons set-forth herein.

III. Chemical Analysis of Sildenafil Citrate and Caffeine in the ODTs:

The analytical determinations used isocratic high-performance liquid chromatography (HPLC) separation on a reversed phase. 70 mg of Sildenafil citrate and 70 mg of caffeine were dissolved in a mobile phase and serially diluted with the mobile phase to give a final working concentration of 70 μg mL⁻¹ of sildenafil citrate and 70 μg mL⁻¹ of caffeine. An Agilent (Germany) series LC system equipped with a degasser, solvent delivery unit and an auto-sampler was used with injections carried out at room temperature. The isocratic mobile phase (pH 4.5) consisted of acetonitrile and (0.05 M) phosphate buffer (30:70 V/V) and 1.0 ml of triethylamine delivered at a flow rate of 1.0 mL min⁻¹ and detection at λ 230 nm.

IV. Pharmacokinetic Study of ODTs: Sildenafil Citrate and an Agent Mixture of Sildenafil Citrate and Caffeine on Healthy Volunteers:

1. Volunteer selection—The clinical trial was performed on six (6) male volunteers between 20 and 40 years of age. All volunteers should have normal physiological examination. The subjects should be without known history of alcohol or drug abuse problems or chronic gastrointestinal, cardiac, vascular, hepatic or renal diseases and should preferably be non-smokers. The suitability of the volunteers would be screened using standard laboratory tests, a medical history, and a physical examination. If necessary, special medical investigations may be carried out before and during studies. Volunteers were excluded from this study if they had evidence of any clinically significant disease or abnormality, including asthma, eczema, drug hypersensitivity and/or a personal or family history of bleeding disorder, migraine or peptic ulceration. The protocol of the study was reviewed and approved by the institutional review board of the Drug Research Center (DRC), Cairo, Egypt. The research was carried out in accordance with the international clinical research guidelines, enunciated in the Declaration of Helsinki, adopted in Helsinki in 1964 and amended in Seoul, South Korea, October 2008. The purpose of this study was fully explained, the informed consent forms were carefully read before signing, and the volunteers gave their written consent. All questions were discussed in detail with the clinical staff. No alcohol or xanthine-containing foods or beverages would be consumed for 48 hrs prior to dosing and until after the last blood sample is collected. Volunteers would take no medications two weeks prior to initiation of the study and until the study is completed. Water may be taken except for 1 hour before and after administration. All meals during the study would be standardized, and the same meals would be served during three phases of the study.

2. Study design—a randomized, single dose, three-way crossover open-label study was performed using (i) the selected sildenafil citrate ODT formula, and (ii) the agent mixture of sildenafil citrate and caffeine ODT formula, as compared to (iii) the conventional market product VIAGRA 50 mg oral tablets (Pfizer, USA). Subjects were hospitalized at drug research center (DRC) the nights before the date of phase I, phase II, and phase III, and during the clinical phase until blood sampling of 12 hours. A signed and dated registration form for each volunteer including time in, time out was applied. Following an overnight fast of at least 10 hours, subjects were administered a single dose of the test or reference product and continued fasting for about 4 hrs after administration of the test and reference treatment. All the volunteers were under complete medical supervision in the DRC.

3. Sample collection—Venous blood samples were collected in heparinized tubes before administration of the dosage form, and sampling time was 0 (pre-dose), 5, 10, 15, 30 and 45 minutes, 1, 1.5, 2, 3, 4, 6, 8 and 12 hours after drug administration. All samples were collected and plasma was immediately separated from blood cells by centrifugation at 3000 rpm for 10 min and stored frozen at −20° C. until analysis.

4. Sample preparation—An appropriate number of disposable glass test tubes was placed in a rack. The tubes were numbered according to the order of the analytical runs, and blanks, and the volunteers human plasma samples (500 ul) were added into appropriate tubes, with the internal standard (50 ul of omeprazole working solution at 500 ng/ml) dispersed and vortexed for 1 min. Then 1 ml of acetonitrile was added to each, vortexed for 1 minute, and the samples were centrifuged at 4000 rpm for 5 minutes, after which a clear supernatant layer was transferred to an auto-sampler vial.

5. Determination of sildenafil citrate and caffeine in human plasma—A sensitive, selective and accurate Liquid Chromatography/Mass Spectrometry/Mass Spectrometry (LC-MS/MS) method was developed and validated (data not shown) using international guidelines before the study for determination of SC and caffeine in human plasma. An omeprazole internal standard (IS) stock solution was prepared by transferring 10 mg of omeprazole into a 100 ml volumetric flask, adding about 80 ml of methanol, sonicating for 10 minutes, and adjusting the volume of solution for a concentration of 100 ug/ml omeprazole. A volume of 0.5 ml of prepared solution was transferred into a 100 ml volumetric flask and the volume was adjusted with water to obtain a concentration of 500 ng/ml omeprazole. The analytical equipment included a liquid chromatograph (Agilent 1200 series, USA) equipped with a degasser; a mass spec detector (Agilent 1200 series Triple Quad, USA); and, an auto-sampler (Agilent 1200 series, USA). 10 ul aliquots of processed samples were injected on Thermo, Hypersil Gold C8, 4.6×50 mm, 5.0 micron. All analyses were carried out at room temperature. The mobile phase consisted of methanol:ammonium formate (70:30) v/v delivered at a flow rate of 0.6 mL/min into the mass spectrometer's electrospray ionization chamber. Quantitation was achieved by MS/MS detection in positive electrospray ionization mode (ESI) for both the sildenafil citrate and internal standard, using the Agilent 6410 mass spectrometer. Ion detection was performed in the multiple reaction monitoring (MRM) mode, monitoring the transition of the m/z 475 precursor ion to the 283 for sildenafil citrate and 346.1 precursor ion to the m/z 197.9 for the internal standard. Analytical data were processed using Mass Hunter, Agilent system software.

6. Monitoring volunteers—Blood pressure was monitored at 0 (pre-dose), 5, 10, 15, 30 and 45 minutes, 1, 1.5, 2, 3, 4, 6, 8 and 12 during the study. Subjects were informed to report any unusual symptoms observed during the study. Subjects were periodically questioned during each phase of the study for any unusual symptoms experienced after drug administration.

7. Pharmacokinetic and statistical analyses—Plasma concentration-time data of sildenafil citrate was analyzed for each subject by non-compartmental pharmacokinetic models using KINETICA software (version 4.4.1). Peak plasma concentrations (Cmax) and the time to peak plasma concentration (Tmax) were directly obtained from the concentration-time data. The area under the plasma concentration-time curve (AUC₀₋₁₂) from time zero to the last time was measured. Relative bioavailability of sildenafil citrate in the ODT and the agent mixture of the sildenafil citrate and caffeine were compared to the commercial product VIAGRA, which was calculated according to the following equation:

Relative bioavailability (%)=AUC₀₋₁₂(oral disintegrating tablets)/AUC₀₋₁₂ (Commercial oral tablets; VIAGRA)×100

Analysis of variance was used to assess the effect of the formulation on pharmacokinetic parameters. Differences between two related parameters were considered statistically significant for p-value equal to, or less than, 0.05.

### EXAMPLE 2

### Performance Testing of Orally Disintegrating Tablets with Sildenafil Citrate

The variety of orally disintegrating tablets prepared in Example 1 were performance tested for (i) uniformity of active agent content, (ii) weight uniformity, (iii) friability, (iv) disintegration time, (v) wetting time, and (vi) moisture analysis.

Physical Characterization of the ODTs:

1. Uniformity of the sildenafil citrate content—The test was carried out according to the European pharmacopoeia (2012) as follows: Ten (10) randomly selected tablets from each formula were individually assayed for drug content uniformity. The mean value of the ten tablets was estimated to calculate the percentage of sildenafil citrate content of the tablets (n=10).

2. Uniformity of weight—The test was carried out according to the European pharmacopoeia (2012) as follows: Twenty (20) tablets from each formula were individually weighed, and the mean of tablet weights was calculated. Not more than two of the individual weights could deviate from the average weight by more than 7.5% and none could deviate by more than twice that percentage.

RESULTS—Table 2 shows that the average weight for ODTs formulae (G1, X1, A1, C1) ranged from (199.2±0.95 mg to 200.7±1.30 mg), meaning that all the tablets fall within the acceptable weight variation range; according to the European pharmacopoeia (2012), not more than two tablets deviated from the average weight by more than 7.5%, and none could deviate by more than twice this percentage.

3. Tablet friability—The test was carried out according to the European pharmacopoeia (2012) as follows: Twenty (20) tablets from each formula were accurately weighed and placed in the drum of friabilator (Erweka tye, GmbH, Germany). The tablets were rotated at 25 rpm for a period of 4 minutes and then removed, dedusted, and accurately re-weighed. The percentage loss in weights was calculated and taken as a measure of friability. The test was run once for each tablet formulation.

RESULTS—Table 2 show the friability results. The ODTs comply with the compendial standards (European pharmacopeia 2012), if the weight loss during the test was less than 1%, and the tablets did not break or show any capping or cracking during the test. The ODTs formulated with gelatin (2%), Na-carboxymethyl cellulose (2%), xanthan gum (2%), and AEROSIL200 (2%) as a matrix former showed a percentage of fines that is within the acceptable range for tablets (less than 1%). Some ODTs were more friable than the others, as they showed higher percentage of weight loss. Tablets formulated with gelatin and AEROSIL200 showed a higher percentage weight loss than those formulated with Na-CMC and xanthan gum. The ODT formulae G1 and A1 showed percentage weight loss of (0.91% and 0.95%) respectively while C1 and X1 ODT formulae showed (0.30% and 0.40%) respectively.

4. In vitro disintegration time—Disintegration times of ODTs were determined using six (6) tablets in distilled water kept at 37±0.5° C. using a disintegration tester (Logan instruments, USA), and discovered to disintegrate in time not more than 3 minutes according to the European pharmacopoeia (2012). The disintegration time was measured as the point in time when there were no particles of tablets or only a trace amount of soft residue remains on the screen. The test results presented are the average of three determinations (n=3).

RESULTS—Table 2 shows the average values of the in vitro disintegration times from the different ODT formulae (G1, X1, A1 and C1). The short disintegration times of the ODTs may have been due to the super disintegrant rapidly taking up water, swelling, and exerting pressure inside tablet to break the tablet into smaller particles that dissolved rapidly. ODTs containing matrix former xanthan gum and Na-CMC have a longer disintegration time compared to tablets containing gelatin and AEROSIL200 as a matrix former. The GI, X1, A1 and C1 ODTs showed average disintegration times of (17±1.00, 20±1.00, 18.6±1.53 and 20±2.00 seconds, respectively. These results were consistent with the results of friability testing where the ODTs containing xanthan gum and Na-CMC as a matrix former are less friable than ODTs containing gelatin and AEROSIL200 as a matrix former. The shortest disintegration times were present with gelatin as matrix former (G1) and disintegrated within 17±1.00 seconds. Addition of xanthan gum increased disintegration times, which may be due to the binding forces of xanthan gum holding the tablet together. The disintegration activity of xanthan gum formula at low concentration, on the other hand, may be due to a greater swelling capacity. And, increasing the disintegration time by adding sodium carboxymethyl cellulose may be due to tremendous swelling capacity of Na-CMC before disintegration occurs.

5. In vivo disintegration time—The oral disintegration time was tested on six (6) healthy volunteers. The protocol of the study was reviewed and approved by the institutional review board of the Drug Research Center, Cairo, Egypt. Before the test, all volunteers received a detailed explanation of the purpose of the study and gave their written consent, selected as having no history of hypersensitivity to sildenafil citrate. Prior to the test, all volunteers were asked to rinse their mouth with distilled water. For the determination of the in vivo disintegration time of the prepared lyophilized tablets, each of the six subjects was given a coded tablet. Tablets were placed on the tongue and the start time was immediately recorded. The subjects were allowed to move the tablet against the upper palate of their mouth with their tongue to cause a gentle tumbling action on the tablet without biting on it or tumbling it from side to side. Immediately after the last noticeable granule had disintegrated, the stop time was recorded. The subjects were asked to spit out the content of their oral cavity after tablet disintegration and rinse their mouth with distilled water. The swallowing of saliva was prohibited during the test, and any remaining saliva was rinsed from their mouths after each measurement. Each subject was asked to test one tablet per day, and the test results are presented as mean value±S.D (n=6).

RESULTS—Table 2 shows the average oral (in vivo) disintegration time of sildenafil citrate ODTs. The in vivo results correlated with in vitro results in that tablets containing xanthan gum and Na-CMC as a matrix former have longer disintegration times than tablets containing gelatin and AEROSIL200 as a matrix former. The ODTs GI, X1, A1 and C1 showed average disintegration times of 15.17±1.04, 18.16±1.04, 16.83±1.76 and 18.67±1.61 seconds, respectively. The in vivo disintegration times were shorter when compared to the in vitro disintegration times, probably due to the gentle movement of the tablet inside the mouth and the gentle mechanical stress on the tablet.

6. Wetting time—Ten milliliters of distilled water containing eosin (a water soluble dye) was placed in a petri dish of 10 cm diameter. The tablet was carefully placed in the center of the petri dish and the time required for the dye to reach the upper surface of the tablet was noted as the wetting time. The test results presented are the average of three determinations (n=3).

RESULTS—Table 2 shows the average wetting times of the different formulae. The wetting times of all tablets were very short. For example, G1, A1, X1, and C1 had wetting times of 8.3±0.58, 8.00±1.73, 9.67±1.53, 9.00±1.00 seconds, respectively, not exceeding 10 seconds. These results correlate with the friability and disintegration time results.

7. Moisture analysis—The tablets were analyzed for their residual moisture content after lyophilization using a Karl Fischer titrator (Veego Matic-MD, Bombay, India). Each tablet was pulverized, inserted in the titration vessel containing dried methanol, and titrated with HYDRANAL Composite 5 reagents after a stirring time of 3 minutes. The test results presented are the average of three determinations (n=3).

RESULTS—Table 2 shows the average percentage moisture content of the ODTs. The residual moisture content in the lyophilized tablets was very small, not exceeding 2% and ranging from 0.9%-1.9%, indicating that lyophilization was efficient in removing water from the prepared ODTs.

TABLE 2

| For- | Drug content | Weight | Friability | In- vitro DT* | In- vivo DT* | Wetting time | Moisture content | Caffeine content |
|---|---|---|---|---|---|---|---|---|
| mula | (%) | (mg) | (%) | (sec.) | (sec.) | (sec.) | (%) | (%) |
| G1 | 99.13 ± 0.21 | 199.2 ± 0.95 | 0.91 | 17 ± 1.00 | 15.17 ± 1.04 | 8.3 ± 0.58 | 0.90 ± 0.10 | — |
| X1 | 98.87 ± 0.81 | 199.3 ± 0.96 | 0.40 | 20 ± 1.00 | 18.16 ± 1.04 | 9.67 ± 1.53 | 1.47 ± 0.64 | — |
| A1 | 99.07 ± 0.15 | 200.7 ± 1.30 | 0.95 | 18.6 ± 1.53 | 16.83 ± 1.76 | 8.00 ± 1.73 | 1.97 ± 0.25 | — |
| C1 | 98.80 ± 1.08 | 199.8 ± 0.36 | 0.30 | 20 ± 2.00 | 18.67 ± 1.61 | 9.00 ± 1.00 | 1.30 ± 0.36 | — |
| G2 | 98.83 ± 0.72 | 199.33 ± 0.57 | 0.87 | 9 ± 1.00 | 9 ± 1.00 | 2 ± 1.00 | 1.10 ± 0.85 | — |
| G3 | 98.87 ± 0.15 | 199.03 ± 0.45 | 0.79 | 9.33 ± 0.52 | 10 ± 1.00 | 2.4 ± .53 | 2.5 ± 0.32 | — |
| G4 | 98.93 ± 0.40 | 199.43 ± 0.61 | 0.68 | 15 ± 1.00 | 15 ± 1.00 | 2.6 ± .58 | 2.6 ± 0.13 | — |
| G5 | 98.60 ± 0.10 | 199.80 ± 0.09 | 0.55 | 16 ± 1.00 | 15.97 ± 0.10 | 5.33 ± 3.05 | 2.7 ± 0.21 | — |
| G6 | 98.97 ± 0.25 | 200.03 ± 0.05 | 0.65 | 15 ± 1.00 | 16 ± 1.00 | 9.17 ± 0.77 | 1.63 ± 0.32 | — |
| X2 | 98.97 ± 0.25 | 200.07 ± 0.42 | 0.44 | 19 ± 1.00 | 19 ± 1.00 | 8 ± 1.00 | 1.60 ± 0.36 | — |
| X3 | 100.30 ± 1.89 | 199.86 ± 0.06 | 0.36 | 18 ± 1.00 | 18 ± 1.00 | 8.7 ± 2.08 | 1.50 ± 0.20 | — |
| X4 | 98.73 ± 0.67 | 200.03 ± 0.12 | 0.49 | 19 ± 1.00 | 19 ± 1.00 | 8.6 ± 3.51 | 1.63 ± 0.32 | — |
| X5 | 98.80 ± 0.44 | 200.03 ± 0.06 | 0.34 | 21 ± 1.00 | 21 ± 1.00 | 9.1 ± 0.76 | 1.23 ± 0.68 | — |
| X6 | 98.87 ± 0.06 | 199.77 ± 0.15 | 0.50 | 22 ± 1.00 | 20 ± 1.00 | 10 ± 1.00 | 1.53 ± 0.38 | — |
| A2 | 98.87 ± 0.49 | 200.10 ± 0.17 | 0.88 | 19 ± 1.00 | 16 ± 2.6 | 5 ± 1.00 | 1.83 ± 1.04 | — |
| A3 | 98.77 ± 1.65 | 200.07 ± 0.10 | 0.82 | 20 ± 1.00 | 16 ± 3.00 | 5.3 ± 1.53 | 2.17 ± 0.15 | — |
| A4 | 98.63 ± 0.12 | 200.03 ± 0.21 | 0.85 | 20 ± 1.00 | 17 ± 3.00 | 5.7 ± 2.52 | 1.97 ± 0.21 | — |
| A5 | 99.50 ± 0.44 | 200.03 ± 0.21 | 0.90 | 17 ± 1.00 | 15.3 ± 2.52 | 6.7 ± 3.51 | 2.13 ± 0.40 | — |
| A6 | 99.70 ± 0.62 | 200.10 ± 0.25 | 0.79 | 19 ± 1.00 | 15.3 ± 4.73 | 7.6 ± 5.13 | 2.13 ± 0.32 | — |
| C2 | 99.27 ± 0.55 | 199.67 ± 0.56 | 0.52 | 23 ± 1.00 | 16.6 ± 1.53 | 10.3 ± 2.52 | 1.47 ± 0.21 | — |
| C3 | 100.2 ± 0.08 | 200.13 ± 0.21 | 0.12 | 17.3 ± 1.53 | 19.0 ± 1.00 | 9 ± 1.00 | 1.13 ± 0.32 | — |
| C4 | 99.63 ± 0.38 | 200 ± 0.26 | 0.34 | 18 ± 2.00 | 18.67 ± 1.53 | 11.67 ± 1.53 | 1.53 ± 0.35 | — |
| C5 | 100.17 ± 0.15 | 199.9 ± 0.171 | 0.50 | 19.3 ± 2.082 | 18.3 ± 0.58 | 9.67 ± 1.52 | 1.03 ± 0.25 | — |
| C6 | 99.83 ± 0.15 | 199.93 ± 0.15 | 0.21 | 19 ± 2.65 | 21 ± 1.00 | 13 ± 5.29 | 1.70 ± 0.20 | — |
| F1 | 98.67 ± 0.13 | 199.74 ± 0.26 | 0.52 | 16.10 ± 0.15 | 15.82 ± 0.07 | 5.00 ± 2.00 | 2.68 ± 0.12 | 99.55 ± 0.34 |

*DT = Disintegration test

8. In vitro dissolution studies—The dissolution profiles of sildenafil citrate in

ODTs compared with the plain drug and market product (VIAGRA) were determined using the USP dissolution tester type II (Pharma Test dissolution Tester, Germany). The amount of drug used was 70 mg sildenafil citrate equivalent to 50 mg sildenafil base. At specified time intervals (1, 2. 3, 5, 7, 10 and 15 min.), 3 ml of dissolution media were withdrawn, and replaced with an equal volume of the fresh medium to maintain a constant total volume. Samples were filtered through a 0.45 um millipore filter and assayed for drug content using HPLC. The cumulative amount of drug dissolved in the preparations was calculated. Dissolution tests were performed three times per formulation (n=3), and the market product, VIAGRA, and sildenafil citrate plain powder were tested in the same way.

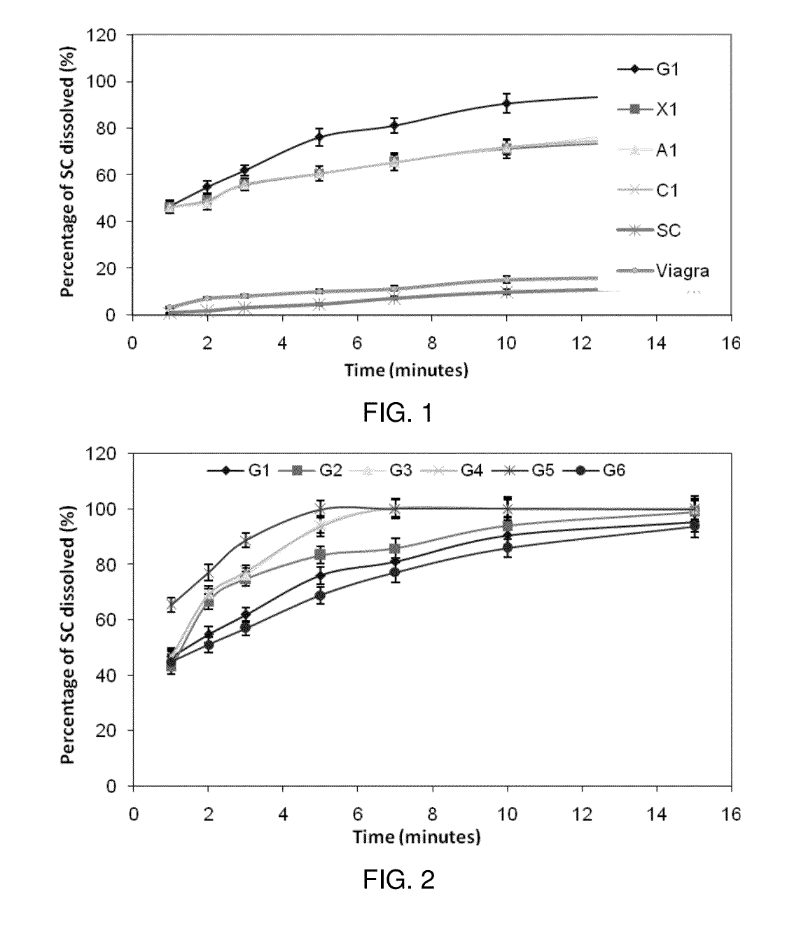

RESULTS—FIG. 1 compares (i) the cumulative sildenafil citrate ODT dissolution as a function of time from formulations G1 (gelatin), X1 (xanthan gum), A1 (AEROSIL200) and C1 (N-CMC) to (ii) sildenafil citrate plain powder and (iii) the market product VIAGRA, according to some embodiments. During first two minutes, the percentage of drug dissolved from formulations G1, X1, A1, C1, the market product, and the plain powder were 54.9%, 49.17%, 47.80%, 49.10%, 7.25%, and 1.8% respectively. The plain powder yielded the slowest dissolution rate, with only 1.8% dissolved after 2 minutes. The hydrophobicity of the powder caused it to float on the surface of the dissolution medium and prevented its surface from contacting the medium. On the other hand, the sildenafil citrate in the lyophilized tablet was immediately dispersed and almost completely dissolved in 15 minutes. The dissolution rate of sildenafil citrate in the lyophilized tablet increased markedly compared to sildenafil citrate powder alone. Results showed that the ODT formulae containing gelatin, xanthan gum, AEROSIL200 and Na-CMC showed a significantly higher dissolution when compared to the sildenafil plain powder and the market product VIAGRA (p<0.05). This may be attributed to the fast disintegration of the tablets and the great improvement in the wettability of sildenafil citrate in the ODT dosage forms.

Formula containing gelatin (G1) showed faster drug release than the corresponding formulae containing AEROSIL200, xanthan gum, and sodium carboxymethyl celloluse (A1, X1 and C1). Statistical analysis revealed that formula containing 2% AEROSIL200 (A1) showed a significant decrease in the percentage of the drug dissolved after two minutes compared to the formula containing 2% gelatin (G1) (P<0.05). This result could be due to increased crushing strength upon addition of AEROSIL200 which slowed down the entrance of dissolution medium into the matrices. Also, statistical analysis revealed that a formulation containing 2% xanthan gum (X1) showed a significant decrease in the percentage of the drug dissolved after two minutes compared to the formulation containing 2% gelatin (G1) (P<0.05). This could be attributed to the hydration of individual xanthan gum particles resulting in extensive swelling and, as a result of the rheological nature of the hydrated matrix, the swollen particles may coalesce. This resulted in a continuous viscoelastic matrix that fills the interstices, maintaining the integrity of the tablet and retarding further penetration of the dissolution medium. Also, statistical analysis revealed that a formulation containing 2% Na-CMC (C1) showed a significant decrease in the percentage of the drug dissolved after two minutes compared to the formula containing 2% gelatin (G1) (P<0.05). This could be because of more polymer entanglement and more gel strength. An increase in gel strength would contribute to a lesser rate of polymer erosion. For all these reasons, the diffusion coefficient of the drug and dissolution through the matrix decreases and results in a lower drug release.

9. The Effect of Adding Stabilizers—

All of the previous formulae (G1, X1, A1 and C1) were further tested in the presence of different solubilizers in an attempt to increase dissolution rate. Five solubilizers were used: PEG (polyethylene glycol) 400, PEG 6000, PVP (polyvinylpyrrolidone) K30 and TWEEN80 (polysorbate 80), each in concentration of 2% w/w. The sildenafil citrate was also complexed with β-cyclodextrin (1:1 molar ratio) to determine the effects of this alternative.

These ODTs were examined in the same way as previously described, including uniformity of weight, friability, drug content uniformity, in vitro disintegration, in vivo disintegration, wetting time, moisture content, and in vitro dissolution. To study the effect of solubilizers on the ODTs, a statistical analysis was performed using a one-way analysis of variance (ANOVA) followed by a multiple comparison procedure (Dunnett's test). The data from the sildenafil citrate oral disintegrating tablets containing gelatin, xanthan gum, AEROSIL200, and Na-CMC as a matrix former, in addition to different solubilizers is presented in Table 2.

The uniformity of the sildenafil citrate content is shown in Table 2. The mean percentage of sildenafil citrate in the ODTs from all formulae containing gelatin, xanthan gum, AEROSIL200, and Na-CMC as a matrix former after addition of solubilizers is high and uniform, ranging from 98.60±0.10% to 100.30±1.89%.

The uniformity of the weight of the sildenafil citrate ODTs is shown in Table 2. The average weight for all tablet formulae after addition of different solubilizers ranged from 199.03±0.45 mg to 200.13±0.21 mg, all tablets of which fall within the acceptable weight variation range, according to the European pharmacopoeia (2012).

The friability results for the prepared sildenafil citrate ODTs are shown in Table 2, ranging from 0.12% to 0.9%. In addition, the tablets did not break or show any capping or cracking during the test. The addition of solubilizers to the different ODTs (G1, X1, A1, C1) did not show any significant effect on friability results (p>0.05).

The in vivo disintegration results are shown in Table 2. The average values of the in vivo disintegration times of different gelatin tablet formulae containing solubilizers were compared to the ODT G1. A statistical analysis revealed that the ODT formulae G2 and G3 showed significantly shorter disintegration times of 9.00±1.00 seconds and 10.00±1.00 seconds compared to the ODT G1 at 15.17±1.04 seconds (p<0.05). On the other hand, the ODTs containing PVPK30, TWEEN80 and sildenafil citrate complexed with β-cyclodextrin (G4, G5 and G6) showed insignificantly shorter in vivo disintegration time compared to the ODT G1 (p>0.05). And, the addition of different solubilizers to X1, A1 and C1 did not affect the in vivo disintegration significantly.

Comparisons of wetting times and moisture analysis results are also shown in Table 2. The average percentage moisture content of different ODT formulae containing solubilizers did not exceed 3% and ranged from 0.81±0.14% to 2.17±0.15% for all formulae, indicating that lyophilization was efficient in removing water from the tablets.

FIG. 2 shows the dissolution profiles of sildenafil citrate from ODTs containing 2% gelatin as a matrix former (G1) and different solubilizers, according to some embodiments. Five different solubilizers were used in the ODTs: (G1) PEG400, (G2) PEG6000, (G3) PVPK30, (G4) TWEEN80, and (G5) β-cyclodextrin, to increase the solubility of sildenafil citrate and increase its dissolution. The PEGs and PVPK30 improve wettability, improving local solubilization and reducing the possibility of forming large particle size drug crystals. TWEEN80 can form micelles to enhance the solubility and increase the rate of release of the drug due to high absorption of the dissolution medium by the tablets and greater disintegration, enhancing drug release and lowering surface tension to make the drug to distribute evenly while preventing a further aggregation of drug particles. Cyclodextrins can form inclusion complexes with many drugs by taking the drug into a central cavity. No covalent bonds are formed or broken during the complex formation and drug molecules in the complex are in rapid equilibrium with free molecules in the solution, increasing its dissolution.

As shown in FIG. 2, the dissolution profiles of sildenafil citrate are from the ODTs containing 2% gelatin as a matrix former (G1) and different solubilizers. Different grades of PEG are used as a solubilizer (G2 and G3) and compared to G1 ODT. At 2 minutes, the percentages of drug dissolved from formula G2 (PEG 400), G3 (PEG 6000) were 66.67±0.57 and 69.43±1.26% respectively, compared to 54.87±0.15% from G1 ODT. It is evident that the addition of PEGs considerably enhanced the rate and extent of the dissolution of the sildenafil citrate.

The addition of PVP K30 to the G1 ODT showed a significant increase in the percentage of drug dissolved after two minutes when compared to the G1 ODT (p<0.05). After two minutes, the percentages of drug dissolved from the G4 ODT (PVP K30) were 68.90±0.78% compared to 54.87±0.15% from the G1 ODT.

The dissolution of the ODT G5 containing TWEEN 80 as a solubilizer showed, after two minutes, a percentage of 77.07±2.10% of sildenafil citrate dissolved compared to 54.87±0.15% from the G1 ODT. The results showed that addition of TWEEN 80 significantly increase the percentage of drug dissolved after 2 minutes when compared to the G1 ODT (p<0.05).

The percentage of drug dissolved from the G6 ODT after two minutes was 51.10±2.77% as compared to 54.987±0.15% from the G1 ODT. The dissolution results showed that complexation of sildenafil citrate with β-cyclodextrin didn't improve the dissolution rate of the drug as compared to the G1 ODT. The percentage of sildenafil citrate dissolved from the lyophilized ODTs after two minutes can be arranged in the descending order as follows G5>G3>G4>G2>G1<G6.

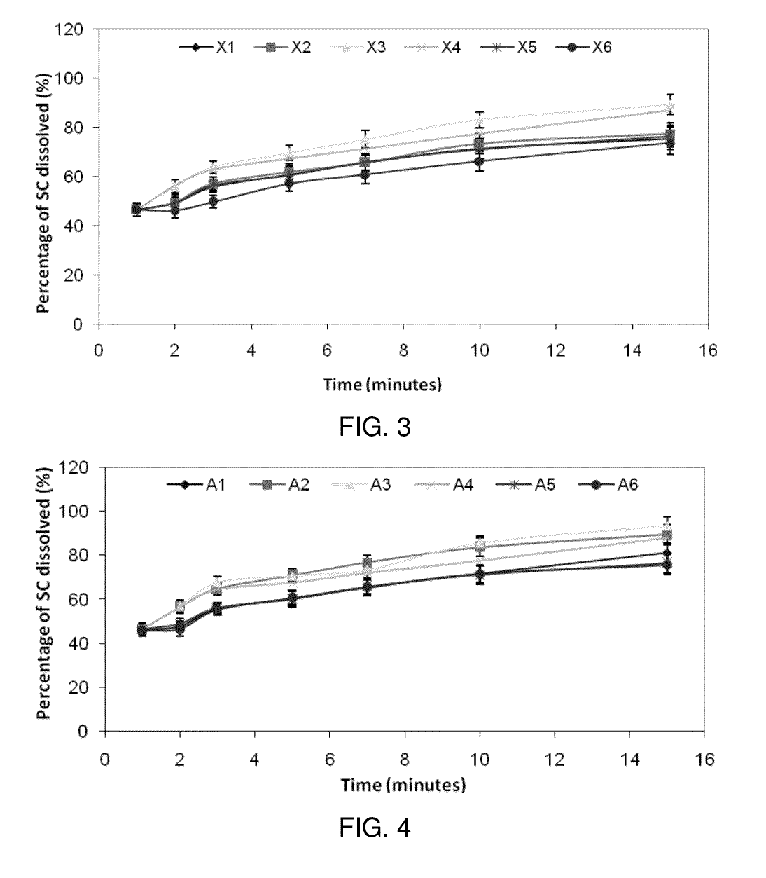

FIG. 3 compares the percentage of sildenafil citrate dissolved from the X1 ODT to ODTs containing 2% xanthan gum as a matrix former and PEG400, PEG6000, and PVPK30 as a solubilizer (X2, X3, and X4), according to some embodiments. After two minutes, the percentages of drug dissolved from formula X2 (PEG 400), X3 (PEG 6000) and X4 (PVPK30) were 49.57±0.81%, 56.00±1.00%, and 56.30±0.98% respectively, as compared to 49.17±0.61% from the X1 ODT. The results showed that the drug dissolution was improved significantly with addition of PEG6000 and PVPK30.

FIG. 4 compares the percentage of sildenafil citrate dissolved from the A1 ODT to ODTs containing 2% AEROSIL200 as a matrix former and PEG400 and PEG6000 as a solubilizer (A2 and A3), according to some embodiments. After two minutes, the percentages of drug dissolved from formula A2 (PEG 400) and A3 (PEG 6000) were 56.53±0.50% and 56.96±0.35%, respectively, as compared to 47.80±2.44% from the A1 ODT. These results showed that the drug dissolution was improved significantly with addition of PEG400 and PEG6000, and this could be attributed to a reduction in particle size of the drug, its deposition on the surface of the carrier, and improved wettability. The percent sildenafil citrate dissolved from the A4 ODT containing PVPK30 as a solubilizer after two minutes was 56.50±1.32%, as compared to 47.80±2.44% from the A1 ODT. These results showed that the drug dissolution was improved significantly with addition of PVPk30. In comparison, the percent sildenafil citrate dissolved from the ODT A5 containing TWEEN 80 as a solubilizer after two minutes was 48.93±1.40%, as compared to 47.80±2.44% from the A1 ODT. The results showed that addition of TWEEN 80 showed no significant difference in the percentage of drug dissolved after two minutes when compared to A1 ODT. The percent sildenafil citrate dissolved from the A6 ODT complexed with β-cyclodextrin was 46.40±0.56%, as compared to 47.80±2.44% from the A1 ODT respectively, showing that complexation of sildenafil citrate with β-cyclodextrin did not improve the dissolution rate of the drug as compared to A1 ODT. The percentage of sildenafil citrate dissolved from the lyophilized ODTs after two minutes can be arranged in the descending order as follows A3>A2>A4>A5>A1<A6.

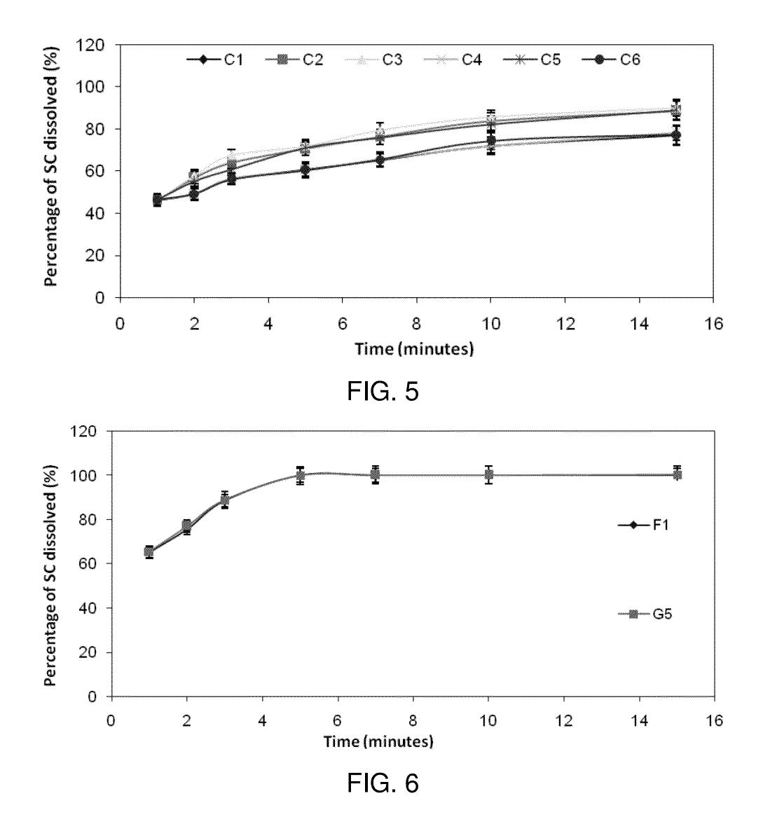

FIG. 5 compares the percentage of sildenafil citrate dissolved from the C1 ODT to ODTs containing shows the percent sildenafil citrate dissolved from the ODTs containing 2% Na-CMC as a matrix former and PEG400 and PEG6000 as a solubilizer (C2 and C3), according to some embodiments. After two minutes, the percentages of drug dissolved from C2 (PEG 400) and C3 (PEG 6000) were 56.86±0.23% and 57.93±1.27%, respectively, as compared to 49.10±0.26% from the C1 ODT. The addition of PEGs significantly increase the drug dissolution rates.

Moreover, after two minutes, the percentage of sildenafil citrate dissolved from the C4 ODTs containing PVPK30 was 49.23±0.35%, as compared to 49.10±0.26% from the C1 ODT. These results showed that addition of PVPK30 to C1 ODTs showed no significant difference in the percentage drug dissolved after two minutes (p>0.05).

Moreover, after two minutes, the percentage of sildenafil citrate dissolved from the ODT C5 containing TWEEN 80 as a solubilizer was 55.33±0.58%, as compared to 49.10±0.26% from the C1 ODT. These results showed that addition of TWEEN 80 showed a significant increase in the percentage of drug dissolved after two minutes when compared to the C1 ODT (p>0.05).

Moreover, the percentage of sildenafil citrate dissolved from the C6 ODT containing SC complexed with β-cyclodextrins was 49.13±1.50%, as compared to 49.10±0.26% from the C1 ODT. A statistical analysis revealed no significant difference in the percentage of drug dissolved after two minutes from C6 ODT as compared to the C1 ODT (p>0.05). The percentage of sildenafil citrate dissolved from the lyophilized ODTs after two minutes can be arranged in the descending order as follows C3>C2>C5>C4>C6<C1.

Given the above results, it is worthy to note that incorporation of gelatin with PEG6000 or PVPK30 or TWEEN 80 in the oral disintegrating tablets together with sildenafil citrate gave a better extent and release rate than other 21 formulae as evidenced by the higher dissolution of G3, G4 and G5 ODTs. According to the above results, G3, G4 and G5 ODTs were chosen for further chemical analyses, such as x-ray diffraction, with their physical mixtures and sildenafil citrate plain powder, as well as further accelerated stability studies.

10. Effect of storage on the prepared sildenafil citrate ODTs (accelerated stability)—Selected tablet formulae were stored in PVC blisters covered with aluminum foil at 75% relative humidity and at 40° C. in a stability cabinet (accelerated stability), during a period of 6 months. Stability was assessed by comparing the results from the drug content, in vitro disintegration, in vivo disintegration, dissolution studies, as well as residual moisture content analysis to fresh prepared sildenafil citrate ODTs. Experiments were done at 0, 1, 3 and 6 months storage. The results were checked for statistical significance using the one-way analysis of variance (ANOVA) to test the equality of several means. A P-value>0.05 was considered statistically insignificant.

RESULTS—Storage at 40° C. and 75% relative humidity for G3, G4 and G5 ODTs showed no significant difference in the mean percentage of sildenafil citrate content with in vitro and in vivo disintegration time during a storage period of six months (p-value<0.05) (data not shown). There was no significant difference in the residual moisture content of sildenafil citrate ODTs G3 and G5 during a storage period of six months (p-value<0.05) (data not shown). On the other hand, the sildenafil citrate ODT G4 showed an increase in the residual moisture content after six months storage (p-value=0.001). For the sildenafil citrate ODTs G3 and G5, no significant difference in the percentage sildenafil citrate dissolved after 1, 2,3, 5,7, 10, 15 minutes during storage for 6 months (p-value>0.05) (data not shown). For sildenafil citrate ODT G4, the percentage of sildenafil citrate that dissolved after two minutes was significantly decreased after storage for six months (p-value=0.019). This appears to be agree with results of the moisture content testing which revealed a significant increase in the moisture content of the tablet during storage. As the moisture uptake by amorphous solids increases, the molecular mobility consequently facilitates the recrystallization process.

Stability studies showed that the sildenafil citrate ODTs G3 and G5 maintained their initial properties with respect to disintegration time, residual moisture, and dissolution characteristics after 6 months storage at 40±2° C. and 75±5% relative humidity. The sildenafil citrate ODT G4 showed significant changes in the residual moisture content and dissolution characteristics during storage. It is worthy to note that, after storage of six months, the percentage of sildenafil citrate that dissolved from the G3 and G5 ODTs after two minutes was 69.30±1.13 and 77.23±1.93, respectively, and after 15 minutes was 99.83±0.28 and 100.00±0.17, respectively. The previous results showed that the ODT G5 is desirable for further in vivo studies.

### EXAMPLE 3

### Performance Testing of Orally Disintegrating Tablets with an Agent Mixture of Sildenafil Citrate and Caffeine

A desirable formulation (G5) set-forth in Examples 1 and 2 was further tested as an agent mixture of sildenafil citrate and caffeine (F1) using the methods of Example 1 and 2. Statistical analyses using independent sample T-tests were done to study the effect of adding caffeine to the orally disintegrating tablets.

1. Uniformity of weight, friability, drug content uniformity, in vitro disintegration, in vivo disintegration, wetting time, and moisture content in the agent mixture—showed no significant difference with the results obtained with sildenafil citrate alone, as shown in Table 2.

2. In vitro dissolution studies for an agent mixture of sildenafil citrate and caffeine—FIG. 6 illustrates dissolution profiles of sildenafil citrate from (i) an ODT with an agent mixture of sildenafil citrate and caffeine (F1) and (ii) the (G5) ODT, according to some embodiments. After two minutes, the percentages of drug dissolved from the F1 ODT was 75.67±1.53, as compared to 77.07±2.10% from the G5 ODT. These results show that addition of caffeine to the G5 ODT showed insignificant effect in the percentage of sildenafil citrate dissolved after two minutes when compared to the G5 ODT (p<0.05). It is evident that the addition of caffeine did not affect the rate and extent of the dissolution of SC from the G5 ODT.

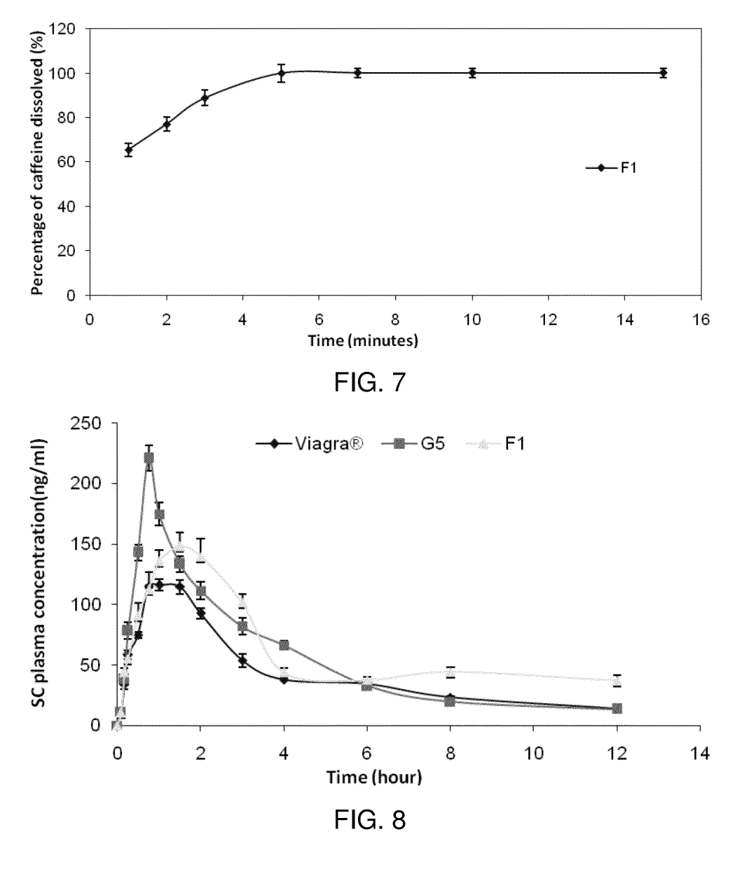

FIG. 7 shows the dissolution profiles of caffeine from the F1 ODT, according to some embodiments. Caffeine has a high dissolution rate and extent with 100% of drug being dissolved after 7 minutes. It is worthy to note that incorporation of caffeine with SC in oral disintegrating tablets together did not affect extent and release rate of SC or the characterization of the oral disintegrating tablets (G5) ODT. According to the above results (F1) ODTs would be introduced for x-ray diffraction with their physical mixtures and SC plain powder and also accelerated stability studies.

### EXAMPLE 4

### Pharmacokinetic Studies in Healthy Volunteers, Comparing the Cmax, Tmax, and AUC₀₋₁₂ in Subjects Taking Various Dosage Forms

This example compares the pharmacokinetic measurements of Cmax, Tmax, and AUC₀₋₁₂ in 6 health volunteers, comparing (i) sildenafil citrate as the only agent in the ODT; (ii) sildenafil citrate in an agent mix with caffeine in the ODT; (iii) caffeine in an agent mix with sildenafil citrate in the ODT; and, (iv) sildenafil citrate as the only agent in the a control tablet that is absorbed through the digestive tract.

For purposes of comparison, VIAGRA is absorbed after oral administration, with a mean absolute bioavailability of 41%. The Tmax ranges from 30 to 120 minutes (median 60 min) from oral dosing in the fasted state. A high-fat meal delays the absorption of sildenafil citrate, with a delay in Tmax and a mean reduction in Cmax of 29%. The sildenafil citrate and its major circulating N-desmethyl metabolite are both approximately 96% bound to plasma proteins, and protein binding is independent of total drug concentration.

FIG. 8 shows the mean plasma concentration versus time curves of sildenafil citrate following administration of the G5 and F1 ODTs as compared to the commercial oral tablets of VIAGRA administered to the human volunteers, according to some embodiments. The corresponding mean pharmacokinetic parameters calculated from the individual curves are collectively summarized in Table 3.

The plasma concentration-time profiles, as well as the calculated pharmacokinetic parameters showed that the G5 ODT improved the oral pharmacokinetic parameters of sildenafil citrate, as expressed by a higher Cmax (1.6 fold), and a shorter Tmax of the G5 ODT, as compared to market product (VIAGRA) with values 0.63 and 1.083 hrs, respectively. Moreover, the AUC₀₋₁₂ of the G5 ODT was higher than that of the market product (VIAGRA), with a surprisingly higher relative bioavailability of 122%. This may be due to the fact that the freeze-drying process imparts a glossy amorphous structure to the bulking agent and, sometimes to the drug, with an increase in the surface area and hence the surface free energy, which result in an increase in the dissolution rate and thereby bioavailability. Also, the plasma concentration-time profiles, as well as the calculated pharmacokinetic parameters, showed that the F1 ODT improved the oral pharmacokinetic parameters of sildenafil citrate, as expressed by a higher Cmax (1.1 fold), as compared to market product (VIAGRA), a shorter Tmax of the F1 ODT, as compared to the market product (VIAGRA) with values of 0.875 hrs and 1.083 hrs, respectively. Moreover, the AUC₀₋₁₂ of the F1 ODT was higher than that of the market product (VIAGRA), with a surprisingly higher relative bioavailability of 125%. As can be seen, the addition of caffeine to the sildenafil citrate ODT did not significantly affect (p>0.05) the pharmacokinetic parameters of sildenafil citrate ODTs (Cmax, Tmax and AUC₀₋₁₂).

TABLE 3

| Pharmacokinetic parameter | SC in G5 | SC in F1 | SC in Viagra ® | Caffeine in F1 |
|---|---|---|---|---|
| Cmax (ng mL−1) | 249.80 ± 129.67 | 177.94 ± 16.99 | 155.34 ± 19.65 | 2210.23 ± 226.84 |
| Tmax (h)** | 0.63 ± 0.10 | 0.88 ± 0.12 | 1.08 ± 0.16 | 0.79 ± 0.07 |
| AUCO-12(ng mL−1 h−1) | 655.03 ± 61.78 | 673.18 ± 38.27 | 536.72 ± 28.44 | 4304.5 ± 451.04 |
| AUCO-∞(ng mL−1 h−1) | 728.47 ± 41.48 | 788.57 ± 57.46 | 609.44 ± 70.62 | 4790.71 ± 399.80 |
| MRTINF | 5.063 ± 0.21 | 6.28 ± 0.48 | 5.89 ± 0.44 | 4.54 ± 0.34 |
| Lz (h−1) | 0.19 ± 0.01 | 0.17 ± 0.01 | 0.19 ± 0.02 | 0.17 ± 0.01 |
| t½ (h) | 3.71 ± 0.15 | 4.26 ± 0.04 | 3.92 ± 0.21 | 4.04 ± 0.37 |
| Average systolic B.P* | 99.00 ± 1.83 | 109.50 ± 1.41 | 100.54 ± 1.13 | — |
| Average diastolic B.P* | 68.67 ± 4.77 | 75.45 ± 1.42 | 68.97 ± 7.21 | — |
| Relative bioavailability (%) | 122% | 125% | — | — |

### EXAMPLE 5

### Pharmacokinetic Studies in Healthy Volunteers, Comparing the Blood Pressure Drops Between Dosage Forms

This example compares the drop in blood pressure in 6 health volunteers, comparing (i) sildenafil citrate as the only agent in the ODT; (ii) sildenafil citrate in an agent mix with caffeine in the ODT; and, (iii) sildenafil citrate as the only agent in the a control tablet that is absorbed through the digestive tract.

The average blood pressure for the volunteers after administration of the ODTs was 109.50±1.41 mmHg over 75.45±1.42 mmHg the F1 ODT; 99.00±1.83 mmHg over 68.67±4.77 mmHg for the G5 ODT; and, 100.54±1.13 mmHg over 68.97±7.21 mmHg for the VIAGRA. These results revealed that addition of caffeine to the sildenafil citrate ODT was effective at inhibiting a substantial decrease in blood pressure caused by administration of both the sildenafil citrate in the G5 ODT and VIAGRA.

TABLE 4

| | | | | Caffeine |
|---|---|---|---|---|
| | SC in G5 | SC in F1 | SC in Viagra ® | in F1 |
| Average | 99.00 ± 1.83 | 109.50 ± 1.41 | 100.54 ± 1.13 | — |
| systolic | | | | |
| B.P* | | | | |
| Average | 68.67 ± 4.77 | 75.45 ± 1.42 | 68.97 ± 7.21 | — |
| diastolic | | | | |
| B.P* | | | | |

Generally speaking, the ODTs taught herein each have physical parameters (content uniformity, weight, friability, in-vitro disintegration time, in-vivo disintegration time, in-vitro dissolution studies and moisture content), were stable over a period of 6 months in accelerated stability studies. The formula of choice, G5, performed with a higher Cmax, a shorter Tmax, and a higher AUC₀₋₁₂, providing an enhanced bioavailability with a rapid onset of action for treatment of erectile dysfunction as compared to VIAGRA. Moreover, the F1 ODT is a surprisingly effective dosage form for sildenafil citrate, for at least the reason that it provides all of the pharmacokinetic benefits of the G5 ODT, in addition to the inhibition of the hypotension side effect of sildenafil citrate.

We claim:

1. A composition comprising a phosphodiesterase type 5 inhibitor (PDE5 inhibitor), or a pharmaceutically acceptable salt thereof, and caffeine, wherein the composition treats erectile dysfunction in a subject while inhibiting a lowering of blood pressure in a subject.

2. The composition of claim 1, wherein the PDE5 inhibitor is sildenafil citrate.

3. A pharmaceutical formulation comprising the composition of claim 1, wherein
the PDE5 inhibitor is sildenafil citrate;
the pharmaceutical formulation is in the form of an oral disintegrating tablet designed for a primary absorption through buccal or sublingual mucosa, the tablet having a matrix former, a sugar alcohol, and a collapse protectant; and,
the oral disintegrating tablet provides a relative bioavailability value for the sildenafil citrate that is substantially greater than a tablet designed for a primary absorption through gastrointestinal mucosa.

4. The pharmaceutical formulation of claim 3, wherein the matrix former is selected from the group consisting of gelatin, xanthan gum, Na-carboxymethyl cellulose, and AEROSIL200.

5. The pharmaceutical formulation of claim 3, wherein the sugar alcohol is selected from the group consisting of mannitol, erythritol, sorbitol, trehalose, xylitol, glucose and sucrose.

6. The pharmaceutical formulation of claim 3, wherein the collapse protectant is selected from the group consisting of gelatin and glycine.

7. The pharmaceutical formulation of claim 3, further comprising a solubilizer selected from the group consisting of polyethylene glycol, polyvinylpyrrolidone, and polysorbate; wherein, the dissolution rate of the sildenafil citrate in the subject being substantially higher than that of a control group receiving the sildenafil citrate without the solubilizer.

8. The pharmaceutical formulation of claim 6, wherein the solubilizer is polyethylene glycol 6000.

9. The pharmaceutical formulation of claim 6, wherein the solubilizer is polyvinylpyrrolidone K30.

10. The pharmaceutical formulation of claim 6, wherein the solubilizer is polysorbate 80.

11. The pharmaceutical formulation of claim 3, wherein the pharmaceutical formulation further comprises a disintegrant selected from the group consisting of croscarmellose sodium, crospovidone, and sodium starch glycoate; wherein, the time to a maximum plasma concentration of the sildenafil citrate in the subject being substantially faster than that of a control group receiving the sildenafil citrate through a commercially available oral tablet configured for a primary absorption through gastrointestinal mucosa.

12. The pharmaceutical formulation of claim 3, further comprising
a solubilizer selected from the group consisting of polyethylene glycol, polyvinylpyrrolidone, and polysorbate; and,
a disintegrant selected from the group consisting of croscarmellose sodium, crospovidone, and sodium starch glycoate; wherein,
the matrix former is selected from the group consisting of gelatin, xanthan gum, Na-carboxymethyl cellulose, and AEROSIL200;
the sugar alcohol is selected from the group consisting of mannitol, erythritol, sorbitol, trehalose, xylitol, glucose and sucrose; and,
the collapse protectant is selected from the group consisting of gelatin and glycine.

13. An article of manufacture comprising:
a pharmaceutical formulation having the composition of claim 1; and,
instructions for administering an effective amount of the pharmaceutical formulation to a subject.

14. A method of treating erectile dysfunction in a subject, comprising administering an effective amount of the composition of claim 1 to the subject, wherein the method inhibits a reduction in blood pressure of the subject while treating the erectile dysfunction.

15. A method of treating erectile dysfunction in a subject, comprising administering an effective amount of the formulation of claim 3 to the subject, wherein the method inhibits a reduction in blood pressure of the subject while treating the erectile dysfunction.

16. A method of treating erectile dysfunction in a subject, comprising administering an effective amount of the formulation of claim 7 to the subject, wherein the method inhibits a reduction in blood pressure of the subject while treating the erectile dysfunction.

17. A method of treating erectile dysfunction in a subject, comprising administering an effective amount of the formulation of claim 11 to the subject, wherein the method inhibits a reduction in blood pressure of the subject while treating the erectile dysfunction.

18. A method of treating erectile dysfunction in a subject, comprising administering an effective amount of the formulation of claim 12 to the subject, wherein the method inhibits a reduction in blood pressure of the subject while treating the erectile dysfunction.

19. The method of claim 18, wherein the time to a maximum plasma concentration (Tmax) of the sildenafil citrate in the subject is substantially faster than that of a control group receiving the sildenafil citrate through a commercially available oral tablet configured for a primary absorption through gastrointestinal mucosa.

20. The method of claim 18, wherein the bioavailability of the sildenafil citrate in the subject is substantially higher than that of a control group receiving the sildenafil citrate through a commercially available oral tablet configured for a primary absorption through gastrointestinal mucosa.

21. A method of making an oral disintegrating tablet for treating erectile dysfunction through a buccal or sublingual absorption, the method comprising:
combining an effective amount of PDE5 inhibitor with an effective amount of caffeine to create an agent mixture;
adding a matrix former, a sugar alcohol, and a collapse protectant to the sildenafil citrate and the caffeine; and,
forming an oral disintegrating tablet that functions to deliver the agent mixture through a buccal or sublingual absorption.

22. The method of claim 21, wherein the PDE5 inhibitor is sildenafil citrate.

23. The method of claim 21, further comprising adding a disintegrant.

24. The method of claim 21, further comprising adding a solubilizer.

25. The method of claim 21, further comprising
adding a solubilizer selected from the group consisting of polyethylene glycol, polyvinylpyrrolidone, and polysorbate; and,
adding a disintegrant selected from the group consisting of croscarmellose sodium, crospovidone, and sodium starch glycoate; wherein,
the matrix former is selected from the group consisting of gelatin, xanthan gum, Na-carboxymethyl cellulose, and AEROSIL200;
the sugar alcohol is selected from the group consisting of mannitol, erythritol, sorbitol, trehalose, xylitol, glucose and sucrose; and,
the collapse protectant is selected from the group consisting of gelatin and glycine.
